SEQUENCE LISTING (34 sequences)

SEQ ID NO: 1 - RNA, 21 nt, Artificial sequence
  Cricetulus griseus sialidase siRNA sense
aucaucugcagggccucgguu

SEQ ID NO: 2 - RNA, 21 nt, Artificial sequence
  Cricetulus griseus sialidase siRNA antisense
ccgaggcccugcagaugauuu

SEQ ID NO: 3 - RNA, 21 nt, Artificial sequence
  Cricetulus griseus sialidase siRNA sense
ggcugccauggaagugugauu

SEQ ID NO: 4 - RNA, 21 nt, Artificial sequence
  Cricetulus griseus sialidase siRNA antisense
ucacacuuccauggcagccuu

SEQ ID NO: 5 - RNA, 21 nt, Artificial sequence
  Cricetulus griseus sialidase siRNA sense
gcagaagacccugcuggccuu

SEQ ID NO: 6 - RNA, 21 nt, Artificial sequence
  Cricetulus griseus sialidase siRNA antisense
ggccagcagggucuucugcuu

SEQ ID NO: 7 - RNA, 21 nt, Artificial sequence
  Cricetulus griseus sialidase siRNA sense
ggagacuaugcuuacagaauu

SEQ ID NO: 8 - RNA, 21 nt, Artificial sequence
  Cricetulus griseus sialidase siRNA antisense
uucuguaagcauagucuccuu

SEQ ID NO: 9 - RNA, 21 nt, Artificial sequence
  Cricetulus griseus sialidase siRNA sense
gccgguccucccuucuccauu

SEQ ID NO: 10 - RNA, 21 nt, Artificial sequence
  Cricetulus griseus sialidase siRNA antisense
uggagaagggaggaccggcuu

SEQ ID NO: 11 - RNA, 21 nt, Artificial sequence
  Cricetulus griseus sialidase siRNA sense
gacggaugagcaugcagauuu

SEQ ID NO: 12 - RNA, 21 nt, Artificial sequence
  Cricetulus griseus sialidase siRNA antisense
aucugcaugcucauccgucuu

SEQ ID NO: 13 - RNA, 21 nt, Artificial sequence
  Cricetulus griseus sialidase siRNA sense
gcaagcuuucccagcaguguu

SEQ ID NO: 14 - RNA, 21 nt, Artificial sequence
  Cricetulus griseus sialidase siRNA antisense
cacugcugggaaagcuugcuu

SEQ ID NO: 15 - RNA, 21 nt, Artificial sequence
  Cricetulus griseus sialidase siRNA sense
gacuaugcuuacagaauccuu

SEQ ID NO: 16 - RNA, 21 nt, Artificial sequence
  Cricetulus griseus sialidase siRNA antisense
ggauucuguaagcauagucuu

SEQ ID NO: 17 - RNA, 21 nt, Artificial sequence
  Cricetulus griseus sialidase siRNA sense
gauuuguuuguccuacgaauu

SEQ ID NO: 18 - RNA, 21 nt, Artificial sequence
  Cricetulus griseus sialidase siRNA antisense
uucguaggacaaacaaaucuu

SEQ ID NO: 19 - RNA, 21 nt, Artificial sequence
  Cricetulus griseus sialidase siRNA sense
cucggacuugcagaacauguu

SEQ ID NO: 20 - RNA, 21 nt, Artificial sequence
  Cricetulus griseus sialidase siRNA antisense
cauguucugcaaguccgaguu

SEQ ID NO: 21 - DNA, 25 bp, Artificial sequence
  Cricetulus griseus sialidase sense primer
cttacagaatccctgctctgatcta

SEQ ID NO: 22 - DNA, 25 bp, Artificial sequence
  Cricetulus griseus sialidase antisense primer
atttgactcatacagacacccaaat

SEQ ID NO: 23 - DNA, 23 bp, Artificial sequence
  Cricetulus griseus sialidase sense primer
gcctcggttaaaagtgagaaaag

SEQ ID NO: 24 - DNA, 21 bp, Artificial sequence
  Cricetulus griseus sialidase antisense primer
aggtaggcttgggtcaccact

SEQ ID NO: 25 - DNA, 21 bp, Artificial sequence
  Synthetic - beta actin sense primer
agctgagagggaaattgtgcg

SEQ ID NO: 26 - DNA, 18 bp, Artificial sequence
  Synthetic - beta actin antisense primer
gcaacggaaccgctcatt

SEQ ID NO: 27 - DNA, 55 bp, Artificial sequence
  Cricetulus griseus S1 shRNA sense
gatccatcatctgcagggcctcggttcaagagaccgaggccctgcagatgatcca

SEQ ID NO: 28 - DNA, 55 bp, Artificial sequence
  Cricetulus griseus S1 shRNA antisense
agcttggatcatctgcagggcctcggtctcttgaaccgaggccctgcagatgatg

SEQ ID NO: 29 - DNA, 55 bp, Artificial sequence
  Cricetulus griseus S5 shRNA sense
gatccgccggtcctcccttctccattcaagagatggagaagggaggaccggctta

SEQ ID NO: 30 - DNA, 55 bp, Artificial sequence
  Cricetulus griseus S5 shRNA antisense
agcttaagccggtcctcccttctccatctcttgaatggagaagggaggaccggcg

SEQ ID NO: 31 - DNA, 49 bp, Artificial sequence
  Cricetulus griseus S1 shRNA
atcatctgcagggcctcggttcaagagaccgaggccctgcagatgatcc

SEQ ID NO: 32 - DNA, 49 bp, Artificial sequence
  Cricetulus griseus S5 shRNA
gccggtcctcccttctccattcaagagatggagaagggaggaccggctt

SEQ ID NO: 33 - DNA, 1366 bp, Artificial sequence
  Cricetulus griseus cytosolic sialidase cDNA in CHO cells
cgggggatggatcatctgcagggcctcggttaaaagtgagaaaagccggtcctcccttctccatttggattacaagctcaaagggactgcttataacctggaggaatgatctccgtgctcccggggggagcactgcccacagtacgtgcatagagaggctttgagaggaattaagatctcaggcccatggcgacttgccctgtcctgcagaaggagacgctattccagacaggagactatgcttacagaatccctgctctgatctacctgtcaaagcagaagaccctgctggcctttgcggaaaagcgtctgaccaagacggatgagcatgcagatttgtttgtcctacgaagaggaagctacaatgcagacacccatcaggtccagtggcaagctgaggaggtggtgacccaagcctacctggagggccaccgctccatgagcccatgtcctttgtatgacaagcagacaaggacccttttccttttcttcatcgctgtccgtgggcaaatatcagaacaccaccagctccagactggggttaatgtcacacggctatgccacatcaccagtactgaccatgggaagacctggagcgctgtccaggaccttacagataccaccattggcagcacccatcaggattgggccacatttggcgtgggtcctgggcactgtctgcagctgcgaaacacagctgggagcctgctggtccctgcttatgcctatcggaaacaaccccctatccatgcacctgccccctctgccttctgcttcctcagccatgaccatgggagcacatgggagctgggccactttgtgtcccagaactcgctggagtgccaggtggctgaggttggcactggcgctgagagggtggtctatctcaatgctaggagctgcctgggagccagggtccaggcacaaagtcctaacagtggcctggatttccaggacaaccaggtagtgagtaaacttgtagagcctcccaaaggctgccatggaagtgtgattgctttccccaaccccacctcaaaggcagatgccttagatgtgtggctgctctatacccaccctacagactcccggaagaggaccaacctgggtgtgtacctcaatcagaagccactggaccccaccacctggtcagctcccaccctgttggcaacaggcatctgtgcctactcggacttgcagaacatggggcacggccctgatggctccccgcaatttgggtgtctgtatgagtcaaataactatgaagagattgttttcctcatgttcaccctgaagcaagctttcccagcagtgtttggtgcccagtgatcttgctgcatgcggcccaaagtgcttcaaaacccccccg

SEQ ID NO: 34 - DNA, 18 bp, Artificial sequence
  Synthetic - pSilencer sequencing primer
aggcgattaagttgggta

(19) United States
(12) Patent Application Publication
(10) Pub. No.: US 2009/0203055 A1
(43) Pub. Date: Aug. 13, 2009

(54) Compositions and methods for RNA interference with sialidase expression and uses thereof

(75) Inventors: Frederyk Ngantung, San Jose, CA (US); Daniel I.C. Wang, Newton, MA (US)

Correspondence Address: CHOATE, HALL & STEWART LLP, TWO INTERNATIONAL PLACE, BOSTON, MA, 02110, US

(73) Assignee: Massachusetts Institute of Technology, Cambridge, MA (US)

(21) Appl. No.: 11/911,878

(22) PCT Filed: Apr. 18, 2006
(86) PCT No.: PCT/US06/14610

§ 371 Date: Aug. 7, 2008

Related U.S. Application Data
(60) Provisional application No. 60/672,350, filed on Apr. 18, 2005.

(51) Int. Cl. C12Q 1/34; C12P 21/06; C12N 5/00; C12N 5/10; C07H 21/02

(52) U.S. Cl. 435/18; 435/69.1; 435/325; 435/353; 435/354; 435/358; 435/363; 435/366; 536/23.2

(57) ABSTRACT

The present invention provides RNAi agents targeted to sialidase. The RNAi agents include siRNA, shRNA, and expression vectors that comprise a template for transcription of an siRNA or shRNA. The invention further provides cells and cell lines that comprise an RNAi agent targeted to sialidase. The cells and cell lines exhibit reduced sialidase activity relative to control cells that do not comprise an RNAi agent targeted to sialidase. Certain of the cell lines stably express the RNAi agent. The invention further provides methods of producing the cells and cell lines. The invention further provides methods for producing a glycoprotein in cells that comprise an RNAi agent targeted to sialidase. The glycoproteins exhibit an improved sialic acid profile relative to glycoproteins produced by cells that do not comprise an RNAi agent targeted to sialidase. The invention further provides glycoproteins, e.g., therapeutic glycoproteins, produced in the cells.

### RELATED APPLICATIONS

This application claims the benefit of U.S. Provisional Application Ser. No. 60/672,350, filed Apr. 18, 2005, the contents of which are incorporated herein by reference in their entirety.

### BACKGROUND OF THE INVENTION

Most eukaryotic secreted proteins, and proteins exposed at the outer surface of the plasma membrane, are glycosylated, i.e., they have one or more sugar groups attached to the polypeptide chain. Such proteins, referred to as glycoproteins, include an increasing number of molecules of diagnostic, therapeutic, and/or industrial interest. Sugar residues found in glycoproteins include mannose, N-acetyl glucosamine, N-acetyl galactosamine, galactose, fucose and sialic acid. Typically these sugars are present as oligosaccharides.

Glycoproteins are commonly produced by expressing genes in recombinant host cells, which are maintained in culture. Eukaryotic cells are generally preferred since prokaryotes lack the enzymes needed for proper glycosylation. One index of protein quality is the number of sialic acid residues capping the oligosaccharide component of a glycoprotein. Sialic acid content is of great importance for various glycoprotein properties, influencing their solubility, thermal stability, and resistance to protease attack (Gu, 1997). Lack of terminal sialic acid can have a number of detrimental effects. For example, it can result in a product which is rapidly removed from the serum by the interaction with receptors in the liver. In proteins such as erythropoietin (EPO) and tissue plasminogen activator (tPA), the presence of terminal sialic acids at the ends of glycans protects them from proteolytic degradation (Goldwasser, 1974; Wittwer, 1990). Chorionic gonadotropin with the presence of terminal sialic acid has a five to seven fold longer residence time in blood compared to asialylated controls (Smith, 1993). These results demonstrate that sialic acid has an important role in preventing glycoproteins from being recognized and cleared by liver asialoglycoprotein receptors (Weiss and Ashwell, 1989). Therefore, the presence of sialic acid as a terminal cap a key determinant of protein quality relates to the presence of sialic acid as a terminal cap on glycans.

Sialidase is a glycohydrolase which cleaves the sialic acid capping the oligosaccharide component of a glycoprotein. Sialidase is often released from cells into tissue culture medium as the cells lose their viability (Gramer, 1995; Munzert, 1996; Gramer, 2000). The latter is a common occurrence for prolonged cell cultivations in both batch and fed-batch processes and can result in a glycoprotein product that exhibits undesirable heterogeneity and impaired bioactivity. Considerable efforts to manipulate culture conditions and/or phenotype have attempted to address this problem (see, e.g., Borys, 1993; Gramer and Goochee, 1993; Wright, 1991; Kimura, 1997; Zanghi, 1999; Hayter, 1992). The use of various chemicals, including sialidase inhibitors, has also been explored (Chotigeat et al, 1994; Chung et al, 2001; Gu et al, 1997). However, there remains a need in the art for methods of improving the sialic acid profile of glycoproteins produced in cell culture systems, e.g., for increasing their sialic acid content. In particular, there remains a need in the art for methods of inhibiting sialidase activity.

### SUMMARY OF THE INVENTION

The present invention provides compositions and methods for inhibiting sialidase expression in a eukaryotic cell. The compositions and methods are based on RNA interference (RNAi). RNAi is an evolutionarily conserved phenomenon in which the presence in a cell of double-stranded RNA containing a portion that is complementary to a target RNA transcript inhibits expression of the target transcript. Inhibition can be caused by cleavage of the transcript or inhibition of its translation. Inhibiting sialidase expression represents a novel approach to controlling sialidase activity.

The invention provides RNAi agents such as short interfering RNA (siRNA) and short hairpin RNA (shRNA) targeted to sialidase. The invention provides additional RNAi agents which are expression vectors that comprise templates for transcription of RNAs that hybridize to each other or self-hybridize to form an siRNA or shRNA that inhibits sialidase expression.

The invention further provides a eukayotic cell comprising an RNAi agent that inhibits sialidase expression. The RNAi agent can be an siRNA, shRNA, or RNAi expression vector. For example, in a preferred embodiment the RNAi agent is an shRNA that comprises a single RNA molecule having complementary regions that hybridize to each other to form a hairpin (stem/loop) structure with a duplex portion approximately 19-29 nucleotides in length and a single-stranded loop. The shRNA may optionally include an unpaired portion that extends beyond the duplex portion (an overhang) at its 5′ or 3′ end. In a preferred embodiment the cell is stably transformed with an RNAi expression vector that comprises a template for transcription of an shRNA. In certain embodiments the cell is a recombinant cell that expresses a protein of interest, e.g., a glycoprotein of interest.

The invention provides a number of different nucleic acids including nucleic acids whose sequences represent target regions of a sialidase transcript, nucleic acids that are strands of an inventive RNAi agent targeted to sialidase, etc. Each such nucleic acid is an aspect of the invention as are RNAi agents that comprise one or more of the sequences or a fragment thereof at least 15 nucleotides in length. Each subsequence or fragment at least 15 nucleotides in length, of a sequence disclosed herein, is an aspect of the invention. Nucleic acids up to 100 nucleotides in length that comprise at least 15 continuous nucleotides of a sequence disclosed herein are additional aspects of the invention.

In another aspect, the invention provides a cell line originating from a cell that stably expresses an RNAi agent that inhibits sialidase expression.

In another aspect, the invention provides a method of generating a cell line comprising: (a) contacting a cell with an RNAi expression vector under conditions suitable for uptake of the vector; (b) isolating single cells that have taken up the vector; and (c) screening populations of cells derived from the single cells of step (b) to identify cells that display reduced sialidase activity relative to parental cells.

In another aspect, the invention provides a method of inhibiting sialidase expression in a cell comprising: contacting the cell with an RNAi agent targeted to a gene that encodes sialidase.

In a related aspect, the invention provides a method of reducing sialidase activity comprising: contacting a cell that expresses sialidase with an RNAi agent targeted to a gene that encodes sialidase. The method reduces sialidase activity in the cell, in medium in which the cell is cultured, or both.

In another related aspect, the invention provides a method of inhibiting sialidase expression in a cell comprising: expressing an RNAi agent targeted to a gene that encodes sialidase in the cell. In a related aspect, the invention provides a method of reducing sialidase activity comprising: contacting a cell that expresses sialidase with an RNAi agent targeted to a gene that encodes sialidase. The method reduces sialidase activity in the cell, in medium in which the cell is cultured, or both.

In another aspect, the invention provides a method of producing a glycoprotein comprising: (a) providing a cell line that expresses a glycoprotein of interest and an RNAi agent targeted to a gene that encodes sialidase; (b) maintaining the cell line for a period of time under conditions suitable for cell growth; and (c) harvesting the glycoprotein of interest.

In another aspect the invention provides a non-human transgenic animal that expresses an RNAi agent targeted to sialidase. The RNAi agent may be, e.g., an siRNA or shRNA.

In another aspect, the invention provides a kit comprising an RNAi agent targeted to sialidase and at least one item selected from the group consisting of: (a) a cell; (b) a transfection reagent; (c) culture medium; (d) a selection agent; (e) an expression vector for insertion of a heterologous protein coding sequence; and (f) instructions for use. The invention further provides a kit comprising a cell that expresses an RNAi agent targeted to sialidase and further comprising at least one item selected from the group consisting of: (a) a transfection reagent; (b) culture medium; (c) a selection agent; (d) an expression vector for insertion of a heterologous protein coding sequence; and (e) instructions for use.

This application refers to various patents, journal articles, and other publications, all of which are incorporated herein by reference. In addition, the following standard reference works are incorporated herein by reference: Ausubel, F. et al. (eds.), Current Protocols in Molecular Biology, Current Protocols in Immunology, Current Protocols in Protein Science, and Current Protocols in Cell Biology, John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001; Goodman and Gilman's The Pharmacological Basis of Therapeutics, 10^{th} Ed. McGraw Hill, 2001.

Unless otherwise defined, all technical and scientific terms used herein have the same meaning as commonly understood by one of ordinary skill in the art to which this invention belongs. For example, the terms “sugar”, “carbohydrate”, “glycan”, “oligosaccharide”, etc., are used as understood in the art. Unless otherwise indicated, nucleic acid sequences are listed in a 5′→3′ direction. Although methods and materials similar or equivalent to those described herein can be used in the practice or testing of the present invention, suitable methods and materials are described below. In the case of conflict, the present specification, including definitions, will control. In addition, the materials, methods, and examples are illustrative only and are not intended to be limiting. Other features and advantages of the invention will be apparent from the following detailed description and claims. Where elements are listed in Markush group format, it is to be understood that each subgroup of these elements is also disclosed, and any element(s) can be removed from the group. Where ranges are given, endpoints are included.

### BRIEF DESCRIPTION OF THE DRAWING

FIG. 1 represents an siRNA as found in Drosophila.

FIG. 2 is a schematic diagram showing steps in RNA interference.

FIG. 3A shows an exemplary siRNA structure.

FIG. 3B shows an exemplary shRNA structure.

FIG. 3C shows an alternative RNAi agent structure.

FIG. 4 shows a translational repression pathway mediated by an RNAi agent that forms an imperfect duplex with a target transcript.

FIG. 5 depicts an example of a construct that may be used to direct transcription of both strands of an siRNA.

FIG. 6 depicts an example of a construct that may be used to direct transcription of a single RNA molecule that hybridizes to form an shRNA in accordance with the present invention.

FIG. 7 shows the action of sialidase on an oligosaccharide component of a glycoprotein.

FIG. 8 shows the nucleotide sequence of a cDNA that encodes hamster cytosolic sialidase (SEQ ID NO: 33).

FIG. 9A shows sialidase mRNA quantification in cells transfected with sialidase siRNA listed in Table 1. Total RNA was isolated from CHO-IFN-γ cells transfected with sialidase siRNA and two step RT-PCR was performed. β-actin mRNA was used as an internal control and the reported sialidase mRNA level from quantitative PCR assay was normalized with β-actin mRNA level.

FIGS. 9B and 9C show an RT-PCR analysis of sialidase mRNA silencing by two different siRNAs. 0.1 Million IFNγ-producing CHO cells were transfected with siRNA Sequence S1 and siRNA Sequence S5, whose DNA sequences are shown in Table 1. RNA was isolated using TRIzol reagent and used for RT-PCR. As shown here, 48-hours post transfection, both Sequence S1 and S5 successfully reduced sialidase mRNA levels. This effect was only transient as sialidase activity gradually increased to its initial level after 72 hours. Lane 1: DNA ladder. Lane 2: PCR product of sialidase cDNA reverse transcribed from RNA of siRNA-transfected cells. The RNA was obtained 48 hours post transfection of sialidase siRNA. Lane 3: PCR product of sialidase cDNA reverse transcribed from RNA of siRNA-transfected cells. The RNA was obtained 72 hours post transfection of sialidase siRNA. Lane 4: PCR product of sialidase cDNA reverse transcribed from RNA of siRNA-transfected cells. The RNA was obtained 96 hours post transfection of sialidase siRNA. Lane 5: PCR product of sialidase cDNA reverse transcribed from RNA of normal cells untransfected with sialidase siRNA.

FIG. 10A is a plot that shows sialidase activity of CHO-IFNγ cells transfected with sialidase siRNA listed in Table 1. CHO-IFNγ cells transfected with sialidase siRNA were collected 48 hours post transfection. 1×10⁶ cells were lysed and the lysate was subjected to fluorescence assay to measure sialidase activity.

FIG. 10B is a plot showing that reduction in sialidase mRNA level by RNAi corresponds to the decrease in sialidase activity. Quantitative PCR analysis and sialidase fluorescence activity assay using methylumbelliferyl substrate were shown to be comparable methods to measure sialidase activity. A decline in sialidase transcript level was shown to correlate with decreased sialidase activity in the CHO-IFN.γ cell lysate.

FIG. 10C shows sialidase activity reduction as a result of transient CHO-IFNγ sialidase mRNA silencing at various time points after siRNA transfection.

FIG. 10D shows sialidase activity reduction as a result of transient CHO-IFNγ sialidase mRNA silencing at various time points after siRNA transfection expressed as % activity reduction.

FIG. 11 is a bar graph showing that siRNA targeted to sialidase effectively reduces sialidase activity in various CHO cell lines.

FIG. 12 is a graph showing that retransfection of CHO cells with sialidase siRNA resulted in restoration of silencing activity. Sialidase siRNA sequence S1 was transfected into CHO-DG44 and CHO-IFNγ at culture times t=0 hours. 1×10⁵ cells were collected for sialidase activity assay every 48 hours. At culture time t=144 hours, each cell line was re-transfected with 5 nmol of sialidase siRNA sequence S1 and 1×10⁵ cells were collected for sialidase activity assay every 48 hours. It was demonstrated that the transient nature of reduction in sialidase activity via the RNAi mechanism can be overcome by continuous delivery of chemically synthesized siRNA.

FIG. 13A is a plot showing that thermodynamic analysis as described by Schwarz, et al. (Schwarz 2003) did not accurately predict the level of reduction in sialidase activity by various siRNAs. The squares (▪) denote sialidase activity measured utilizing fluorescence assay as compared to estimated asymmetrical thermodynamic parameters.

FIG. 13B is a plot showing thermodynamic analysis as described by Khvorova, et al. (Reynolds 2004). The average of internal stability (AIS) analysis for positions 9-14 of the antisense strand (AIS) prediction yielded the opposite trend to that shown by experimental results. Average of internal stability values for positions 9-14 of the antisense strand (AIS) was calculated as described (Reynolds 2004). Based on such analysis, it would predicted that a double stranded RNA bearing less negative AIS should yield a more potent silencing effect. In our experiment, we observed the opposite trend.

FIG. 14 is a graph showing the sialidase activity profile for stably transfected clonal CHO cell lines. Sequence S1 and S5 were incorporated into separate plasmids used to create stable cell lines that continuously produced sialidase siRNA. Clones S1E (which was transfected with a plasmid encoding siRNA sequence S1) and S5F (which was transfected with a plasmid encoding sequence S5) were found to maintain low sialidase activity throughout various phases of batch cell culture. On the other hand, clone S5B showed inconsistent sialidase reduction: during the growth phase and the death phase of cell culture, it exhibited sialidase activity similar to that of the parent cell.

FIG. 15 is a graph showing a stability analysis of sialidase suppression on stably transfected CHO cell clones. S1E cell lines and parental cell lines were analyzed during their growth phase over multiple generations. It was found that the reduction in sialidase activity possessed by the S1E cell line was maintained over 25 generations; indicating the stability of sialidase suppression.

FIG. 16 is a graph showing viable cell densities of stably transfected clones and the parental cell line over time. Cell enumeration was performed throughout cell culture over numerous passages. It was observed that cells transfected with an siRNA-producing plasmid exhibited similar growth profile to that of parental cells, as shown by similar length of lag phase, growth phase, and death phase.

FIG. 17 is a graph showing glycoprotein (IFNγ) titer produced by a stably transfected clone compared with that of the parental cell line. The amount of IFNγ was quantified throughout cell culture by ELISA. It was found that stably transfected cells producing an shRNA targeted to sialidase produced IFNγ in an amount comparable to parent cells.

FIG. 18 is a graph showing the sialic acid content of a glycoprotein (IFNγ) produced by a stably transfected clone producing an shRNA targeted to sialidase compared with that of the parental cell line. In the parental cell line, as culture time increased, CHO cells began to die and sialidase was released into the supernatant. As a result, the amount of sialic acid per glycoprotein decreased as a function of increasing culture time. In contrast, for the S1E cell line, although the number of viable CHO cells gradually decreased, the sialic acid content of IFNγ remained constant, demonstrating the effectiveness of siRNA targeted to sialidase in increasing the sialic acid content of glycoproteins.

FIGS. 19A and 19B show glycan site occupancy of IFNγ from a CHO cell clone that stably expresses an shRNA targeted to sialidase, compared with that for the parental cell line. There are two potential sites for N-linked glycosylation in IFNγAsn²⁵ and Asn⁹⁷. Therefore, IFNγ can be either occupied at both sites (2N), one site (1N), or unoccupied (0N). We showed that there were no significant differences in glycan site occupancy of IFNγ produced by clone S1E and the parental cell line throughout the period over which the assays were performed.

### ABBREVIATIONS

DNA: deoxyribonucleic acid

RNA: ribonucleic acid

mRNA: messenger RNA transcribed from cellular genes, positive strand, a template for protein synthesis

dsRNA: double-stranded RNA

siRNA: short interfering RNA

shRNA: short hairpin RNA

miRNA: microRNA

RNAi: RNA interference

bp: base pair(s)

nt: nucleotide(s)

### DEFINITIONS

The term “antibody”, as used herein, refers to an immunoglobulin, which may be natural or wholly or partially synthetically produced in various embodiments of the invention. An antibody may be derived from natural sources (e.g., purified from a rodent, rabbit, chicken (or egg) from an animal that has been immunized with an antigen or a construct that encodes the antigen) partly or wholly synthetically produced. An antibody may be a member of any immunoglobulin class, including any of the human classes: IgG, IgM, IgA, IgD, and IgE. The antibody may be a fragment of an antibody such as an Fab′, F(ab′)₂, scFv (single-chain variable) or other fragment that retains an antigen binding site, or a recombinantly produced scFv fragment, including recombinantly produced fragments. See, e.g., Allen, T., Nature Reviews Cancer, Vol. 2, 750-765, 2002, and references therein. Preferred antibodies, antibody fragments, and/or protein domains comprising an antigen binding site may be generated and/or selected in vitro, e.g., using techniques such as phage display (Winter, G. et al. 1994. Annu. Rev. Immunol. 12:433-455, 1994), ribosome display (Hanes, J., and Pluckthun, A. Proc. Natl. Acad. Sci. USA. 94:4937-4942, 1997), etc. An antibody may be a “humanized” antibody in which, for example, a variable domain of rodent origin is fused to a constant domain of human origin, thus retaining the specificity of the rodent antibody. The domain of human origin need not originate directly from a human in the sense that it is first synthesized in a human being. Instead, “human” domains may be generated in rodents whose genome incorporates human immunoglobulin genes. See, e.g., Vaughan, et al., Nature Biotechnology, 16: 535-539, 1998. An antibody may be polyclonal or monoclonal, though for purposes of the present invention monoclonal antibodies are generally preferred.

The terms “approximately” or “about” in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

The term “complementary” is used herein in accordance with its art-accepted meaning to refer to the capacity for precise pairing between particular bases, nucleosides, nucleotides or nucleic acids. For example, adenine (A) and uridine (U) are complementary; adenine (A) and thymidine (T) are complementary; and guanine (G) and cytosine (C), are complementary and are referred to in the art as Watson-Crick base pairings. If a nucleotide at a certain position of a first nucleic acid sequence is complementary to a nucleotide located opposite in a second nucleic acid sequence, the nucleotides form a complementary base pair, and the nucleic acids are complementary at that position. A percent complementarity of two nucleic acids within a window of evaluation may be evaluated by determining the total number of nucleotides in both strands that form complementary base pairs within the window, dividing by the total number of nucleotides within the window, and multiplying by 100. The two nucleic acid are aligned in anti-parallel orientation for maximum complementarity over the window, allowing introduction of gaps. When computing the number of complementary nucleotides needed to achieve a particular % complementarity, fractions are rounded to the nearest whole number. A position occupied by non-complementary nucleotides constitutes a mismatch. Typically a degree of complementarity is determined over a window of evaluation at least 15 nt in length, e.g., 19-29 nt.

An “effective amount” of an active agent refers to the amount of the active agent sufficient to elicit a desired biological response. As will be appreciated by those of ordinary skill in this art, the absolute amount of a particular agent that is effective may vary depending on such factors as the desired biological endpoint, the agent to be delivered, the target cell type, etc. For example, an effective amount of an RNAi agent may be an amount sufficient to achieve one or more of the following: (i) reduce expression of a sialidase transcript by at least 2-fold, preferably at least 10-fold, more preferably at least 100-fold; (ii) reduce sialidase activity by at least 25%, preferably at least 50%, e.g., 60%, 70%, 80%, 90%, or more; (iii) increase sialic acid content of a glycoprotein produced by a cell or cell culture by at least 10%, at least 20%, at least 30%, or more over a predetermined time period (e.g., 24-200 hours, or any subrange thereof) or culture phase (e.g., growth phase, lag phase, death phase or combinations thereof), etc.

“Exogenous” or “heterologous” refers to a substance, e.g., a nucleic acid, polypeptide, etc., that originates outside a cell of interest or is not native to the cell in the form in which it presently occurs there. For example, if a host cell is transformed with a nucleic acid sequence that does not occur in the untransformed host cell, that nucleic acid sequence is said to be heterologous with respect to the host cell. The transforming nucleic acid may include a heterologous promoter, heterologous coding sequence, and/or heterologous termination sequence. The transforming nucleic acid may be completely heterologous or may include any possible combination of heterologous and endogenous nucleic acid sequences. Similarly, heterologous refers to a nucleotide sequence derived from and inserted into the same natural, original cell type, but which is present in a non-natural state, e.g., a different copy number, or under the control of different regulatory elements. If a nucleic acid is introduced into a cell, any nucleic acid that is subsequently derived from the nucleic acid (e.g., following one or more rounds of replication, transcription, etc.) is considered exogenous even though it is synthesized in the cell. A polypeptide that is translated from an exogenous nucleic acid is considered exogenous. Any nucleic acid or polypeptide whose expression in a cell is engineered by the hand of man using recombinant DNA technology is considered exogenous.

An “expression vector” is a vector that contains regulatory sequences (e.g., promoters and/or other expression signals and, optionally, 3′ sequences, such as 3′ regulatory sequences or termination signals sufficient to drive transcription of nucleic acid segment to which they are operably linked. The expression vector may also comprise operably linked sequences required for proper translation of the nucleic acid segment. The nucleic acid segment may, but need not be, a protein coding sequence. The nucleic acid segment may be chimeric, meaning that it includes more than one sequence of distinct origin that are fused together by recombinant DNA techniques, resulting in a nucleotide sequence that does not occur naturally. The term “expression vector” can refer to a vector either before or after insertion of the operably linked nucleic acid segment.

The term “gene”, as used herein, has its meaning as understood in the art. In general, a gene is taken to include gene regulatory sequences (e.g., promoters, enhancers, etc.) and/or intron sequences and typically also includes a sequence that encodes a polypeptide (open reading frames). It will be appreciated that a “gene” can refer to a nucleic acid that does not encode proteins but rather encodes a functional RNA molecule.

A “gene product” or “expression product” is an RNA transcribed from the gene (e.g., either pre- or post-processing) or a polypeptide encoded by an RNA transcribed from the gene (e.g., either pre- or post-modification).

A “glycoprotein” is a polypeptide that has one or more carbohydrate moieties covalently attached to it.

The term “hybridize”, as used herein, refers to the interaction between two nucleic acid sequences comprising or consisting of complementary portions such that a duplex structure is formed that is stable under the particular conditions of interest, e.g., in a eukaryotic cell, in a Drosophila lysate, etc. One of ordinary skill in the art will recognize that a first nucleic acid is typically considered to hybridize to a second nucleic acid if the Tm of a duplex formed by the first and second nucleic acids is less than 15° C. below, preferably less than 10° C. below the Tm of a duplex that would be formed by the second nucleic acid and a third nucleic acid that is the same length as, and 100% complementary to, the second nucleic acid and contains nucleosides and internucleosidic linkages of the same type. The Tm is defined as the temperature at which 50% of a nucleic acid and its perfect complement are in duplex in solution. One of ordinary skill in the art will be able to calculate Tm values. A number of empirical formulas are available for so doing, any of which could be used since the fact that a comparison is being made is more important than the accuracy of any particular formula. Several studies have derived accurate equations for Tm using thermodynamic basis sets for nearest neighbor interactions. Values for thermodynamic parameters are available in the literature. For RNA see Freier, S. M., et al., Proc. Natl. Acad. Sci. 83, 9373-9377, 1986. Rychlik, W., et al., Nucl. Acids Res. 18(21), 6409-6412, 1990. Preferably the more recent values and methods in Walter, A. E., Proc. Natl. Acad. Sci., 91, 9218-9222, 1994, or more preferably those in Mathews, D H, J. Mol. Biol., 288, 911-940, 1999, are used. Computer programs for calculating Tm are widely available. See, e.g., the Web site having URL www.basic.nwu.edu/biotools/oligocalc.html. Preferably a program for calculating relevant parameters such as ΔG and Tm, available on the mfold web server at the URL www.bioinfo.rpi.edu/applications/mfold, as described in Zuker, M., Nucl. Acids. Res., 31(13), 2003 is used. Specific hybridization conditions suitable for various applications are known in the art and/or found in standard reference works, e.g., Ausubel, supra, and Sambrook, supra.

“Identity” refers to the extent to which the sequence of two or more nucleic acids is the same. The percent identity between first and second nucleic acids over a window of evaluation may be computed by aligning the nucleic acids in parallel orientation, determining the number of nucleotides within the window of evaluation that are opposite an identical nucleotide allowing the introduction of gaps to maximize identity, dividing by the total number of nucleotides in the window, and multiplying by 100. When computing the number of identical nucleotides needed to achieve a particular % identity, fractions are to be rounded to the nearest whole number. Typically the window of evaluation is at least 15 nt in length, e.g., 19 nt, where the length does not include gaps.

“Inhibit expression of a gene” means to reduce the amount of an expression product of the gene. The expression product can be an RNA transcribed from the gene (e.g., an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein. Such methods include Northern blotting, in situ hybridization, RT-PCR, sequencing, immunological methods such as immunoblotting, immunodetection, or fluorescence detection following staining with fluorescently labeled antibodies, oligonucleotide or cDNA microarray or membrane array, protein array analysis, mass spectrometry, etc. In the case of a protein having a known biological or biochemical activity, an assay of the activity (e.g., in a purified preparation, cell lysate, cell, organism, etc.) can serve as an indicator of expression level.

“In vivo”, as used herein with respect to the synthesis, processing, or activity of an RNAi agent generally refers to events that occur within a cell as opposed to in a cell-free system. In general, the cell can be maintained in tissue culture or can be part of an intact organism.

“Isolated”, as used herein, means 1) separated from at least some of the components with which it is usually associated in nature; 2) prepared or purified by a process that involves the hand of man; and/or 3) not occurring in nature. Any of the nucleic acid and nucleic acid structures described herein may be in isolated form.

A “nucleic acid”, also referred to as a “polynucleotide”, is a polymer of nucleosides. Any of the nucleic acids disclosed herein may be in DNA or RNA form and may be single or double-stranded. Where a nucleic acid sequence is presented, the complementary sequence is also disclosed.

“Nucleobase”, as used herein, means a nitrogen-containing heterocyclic moiety capable of forming hydrogen bonds, preferably Watson-Crick hydrogen bonds, in pairing with a complementary nucleobase or nucleobase analog, e.g., a purine or a pyrimidine. Typical nucleobases are the naturally occurring nucleobases adenine, guanine, cytosine, uracil, thymine, and analogs of the naturally occurring nucleobases. See, e.g., (Fasman, Practical Handbook of Biochemistry and Molecular Biology, pp. 385-394, CRC Press, Boca Raton, Fla., 1989). The terms “nucleobase” and “base” are used interchangeably herein.

“Operably linked”, as used herein, refers to a relationship between two nucleic acids sequences wherein the expression of one of the nucleic acid sequences is controlled by, regulated by, modulated by, etc., the other nucleic acid sequence. For example, the transcription of a nucleic acid sequence is directed by an operably linked promoter sequence; post-transcriptional processing of a nucleic acid is directed by an operably linked processing sequence; the translation of a nucleic acid sequence is directed by an operably linked translational regulatory sequence; the transport or localization of a nucleic acid or polypeptide is directed by an operably linked transport or localization sequence; and the post-translational processing of a polypeptide is directed by an operably linked processing sequence. Preferably a nucleic acid sequence that is operably linked to a second nucleic acid sequence is covalently linked, either directly or indirectly, to such a sequence, although any effective three-dimensional association is acceptable.

“Polypeptide”, as used herein, refers to a polymer of amino acids. A protein is a molecule composed of one or more polypeptides. The terms “protein”, “polypeptide”, and “peptide” may be used interchangeably. Polypeptide may refer to an individual polypeptide or a collection of peptides. Polypeptides as described herein preferably contain only natural amino acids, although non-natural amino acids (i.e., compounds that do not occur in nature but that can be incorporated into a polypeptide chain) and/or amino acid analogs as are known in the art may alternatively be employed.

“Purified”, as used herein, means separated from many other compounds or entities. A compound or entity may be partially purified, substantially purified, or pure, where it is pure when it is removed from substantially all other compounds or entities (other than solvents, ions, etc.), i.e., it is preferably at least about 90%, more preferably at least about 91%, 92%, 93%, 94%, 95%, 96%, 97%, 98%, 99%, or greater than 99% pure. A partially or substantially purified compound or entity may be removed from at least 50%, at least 60%, at least 70%, or at least 80% of the material with which it is naturally found, e.g., cellular material such as cellular proteins and/or nucleic acids. In a preferred embodiment a purified protein is removed from at least 90%, preferably at least 95%, more preferably at least 99%, or more, of the other proteins in a preparation, so that the purified protein constitutes at least 90%, preferably at least 95%, more preferably at least 99%, of the material in the preparation on a dry w/w basis.

“Recombinant host cells”, “host cells”, “cells”, “cell lines”, “cell cultures”, and other such terms denote prokaryotic or eukaryotic cells cultured as unicellular entities and refer to cells which can be, or have been, used as recipients for a recombinant nucleic acid (e.g., a vector) or other transferred nucleic acid (typically DNA). Typically the nucleic acid comprises a template for transcription of a polynucleotide within the cell. The term includes the progeny of the original cell into which the vector or other nucleic acid has been introduced. The cells may be immortalized, i.e, able to undergo a large and indefinite number of divisions in culture under appropriate conditions.

A “recombinant nucleic acid” refers to a nucleic acid that comprises at least two portions that are not naturally found in that configuration, e.g., are not found attached to one another in that particular order in nature. For example, the two portions may originate from different organisms. The portions can be, for example, regulatory sequences, polypeptide coding sequences, etc. Recombinant nucleic acids are typically produced using methods well known in the art and described, for example, in Ausubel, supra, and Sambrook, supra.

A “recombinant polypeptide” refer to a polypeptide produced by expression of a coding sequence found in a recombinant nucleic acid. Typically the recombinant polypeptide is produced in a cell in an amount different from that occurring in nature, e.g., the recombinant polypeptide may be produced in a cell that does not naturally produce that polypeptide.

The term “regulatory sequence” is used herein to describe a region of nucleic acid sequence that directs, enhances, or inhibits the expression (particularly transcription, but in some cases other events such as splicing or other processing) of sequence(s) with which it is operatively linked. The term includes expression signals such as promoters, enhancers and other transcriptional control elements. In some embodiments of the invention, regulatory sequences may direct constitutive expression of a nucleotide sequence; in other embodiments, regulatory sequences may direct inducible expression. The regulatory sequence may comprise a promoter and/or enhancer that is active in a cell type of interest and/or under conditions of interest, e.g., an inducible promoter. For example, expression may be induced by the presence or addition of an inducing agent such as a hormone or other small molecule, by an increase in temperature, etc.

The term “RNAi agent” is used to refer to a species such as an siRNA, shRNA, or other nucleic acid structure that can be processed intracellularly to yield an inhibitory agent such as an siRNA that inhibits expression of a target transcript by RNA interference. The term also includes RNAi expression vectors. An RNAi agent of the invention is modified or generated by the hand of man, or is present in its current location as a result of human intervention as opposed, for example, to an endogenous RNA species. The term “short RNAi agent” is used to refer to relatively small RNAi agents such as siRNA, shRNA, etc., that form or contain a duplex structure under approximately 100 nucleotides in length, typically under approximately 30 nucleotides in length.

An “RNAi expression vector” is a vector that comprises a nucleic acid that serves as a template for transcription of one or more RNAs that self-hybridize or hybridize to each other to form an siRNA or shRNA or other small RNA species that can be processed intracellularly or, optionally, in a cell lysate such as a Drosophila lysate, to yield an agent that inhibits expression of a target gene by RNA interference. The nucleic acid template is operably linked to expression signal(s), e.g., a promoter and optionally 3′ sequences, such as 3′ regulatory sequences or termination signals, so that transcription occurs when the vector is introduced into host cell. The RNAi expression vector may be a DNA or RNA plasmid, a virus, etc. Preferably the RNAi expression vector is a plasmid. Presence of a viral genome in a cell is considered to constitute presence of the virus within the cell, and a vector is considered to be present within a cell if it is introduced into the cell, enters the cell, or is inherited from a parental cell, regardless of whether it is subsequently modified or processed within the cell. The vector, or a portion thereof, may integrate into the host cell genome or may be maintained as an episome. An RNAi expression vector may be used for a variety of purposes in addition to transcript inhibition in a cell. For example, they may be used for in vitro production of an RNAi agent such as an siRNA or shRNA and/or for production of the agent in a cell that may or may not contain a transcript to which the vector is targeted.

A “short, interfering RNA” comprises a double-stranded (duplex) RNA that is between 15 and approximately 29 nucleotides in length or any other subrange or specific value within the interval between 15 and 29, e.g., 16-18, 17-19, 21-23, 24-27, 27-29 nt long and optionally further comprises one or two single-stranded overhangs, e.g., a 3′ overhang on one or both strands. In certain embodiments the duplex is approximately 19 nt long. The overhang may be, e.g., 1-6 residues in length, e.g., 2 nt. An siRNA may be formed from two RNA molecules that hybridize together or may alternatively be generated from an shRNA. In certain embodiments of the invention one or both of the 5′ ends of an siRNA has a phosphate group while in other embodiments one or more of the 5′ ends lacks a phosphate group. In certain embodiments of the invention one or both of the 3′ ends has a hydroxyl group while in other embodiments they do not. One strand of an siRNA, which is referred to as the “antisense strand” or “guide strand” includes a portion that hybridizes with a target transcript. In certain preferred embodiments of the invention, the antisense strand of the siRNA is 100% complementary with a region of the target transcript, i.e., it hybridizes to the target transcript without a single mismatch or bulge over a target region between 15 and approximately 29 nt in length, preferably at least 16 nt in length, more preferably 18-20, e.g., 19 nt in length. The region of complementarity may be any subrange or specific value within the interval between 17 and 29, e.g., 17-18, 19-21, 21-23, 19-23, 24-27, 27-29. In other embodiments the antisense strand is substantially complementary to the target region, i.e., one or more mismatches and/or bulges exists in the duplex formed by the antisense strand and a target transcript. The two strands of an siRNA are substantially complementary, preferably 100% complementary to each other within the duplex portion.

The term “short hairpin RNA” refers to an RNA molecule comprising at least two complementary portions hybridized or capable of hybridizing to form a double-stranded (duplex) structure sufficiently long to mediate RNAi (as described for siRNA duplexes), and at least one single-stranded portion that forms a loop connecting the regions of the shRNA that form the duplex. The structure is also referred to as a stem/loop structure, with the stem being the duplex portion. The structure may further comprise an overhang (e.g., as described for siRNA) on the 5′ or 3′ end. Preferably, the loop is about 1-20, more preferably about 4-10, and most preferably about 6-9 nt long and/or the overhang is about 1-20, and more preferably about 2-15 nt long. The loop may be located at either the 5′ or 3′ end of the region that is complementary to the target transcript whose inhibition is desired (i.e., the antisense portion of the shRNA). In certain embodiments the overhang comprises one or more U residues, e.g., between 1 and 5 Us. As described further below, shRNAs are processed into siRNAs by the conserved cellular RNAi machinery. Thus shRNAs are precursors of siRNAs and are, in general, similarly capable of inhibiting expression of a target transcript that is complementary to a portion of the shRNA (referred to as the antisense or guide strand of the shRNA). In general, the features of the duplex formed between the antisense strand of the shRNA and a target transcript are similar to those of the duplex formed between the guide strand of an siRNA and a target transcript. In certain embodiments of the invention the 5′ end of an shRNA has a phosphate group while in other embodiments it does not. In certain embodiments of the invention the 3′ end of an shRNA has a hydroxyl group while in other embodiments it does not.

An RNAi agent is considered to be “targeted” to a target transcript for the purposes described herein if the RNAi agent comprises a strand that is at least 80%, preferably at least about 85%, 90%, 91%, 92%, 93%, 94%, 95%, 96%, 97%, 98%, 99%, or 100% complementary with the target transcript over a region of at least about 15, more preferably at least about 17, yet more preferably at least about 18 or 19 to about 21-23, or 24-29 continuous nt; and/or one strand of the RNAi agent hybridizes to the target transcript under stringent conditions for hybridization of small (<50 nucleotide) RNA molecules in vitro and/or under conditions typically found within the cytoplasm of mammalian cells or in a Drosophila lysate as described herein. For example, the Tm of duplex formed by the antisense strand of the RNAi agent and a target transcript may be up to 15° C. lower, more preferably up to 10° C. lower, or up to 5° C. lower than the Tm of a duplex formed by the antisense strand of the RNAi agent and a transcript that is 100% complementary to it over a region of equivalent length, e.g., at least about 15, more preferably at least about 17, yet more preferably at least about 18 or 19 to about 21-23, or 24-29 continuous nt. An RNAi expression vector is considered to be targeted to a transcript if presence of the vector within a cell results in production of one or more RNAs that hybridize to each other or self-hybridize to form an siRNA, shRNA, or other short RNA species that can be processed intracellularly to yield an agent that inhibits expression of a target gene by RNAi interference. Since the effect of an RNAi agent that is targeted to a transcript is to reduce or inhibit expression of the gene that directs synthesis of the transcript, an RNAi agent targeted to a transcript is also considered to target the gene that directs synthesis of the transcript. For purposes of description, “targeted to sialidase”, “targeted to a gene that encodes sialidase”, “targeted to a sialidase transcript”, and other similar expressions are considered equivalent unless otherwise evident from the context.

A “target region” is a region of a target transcript that hybridizes with an antisense strand of an RNAi agent.

A “target transcript” is any RNA that is a target for inhibition by RNA interference. The terms “target RNA” and “target transcript” are used interchangeably herein.

The term “vector” refers to a nucleic acid molecule capable of mediating entry of, e.g., transferring, transporting, etc., a second nucleic acid molecule into a cell. The transferred nucleic acid is generally linked to, e.g., inserted into, the vector nucleic acid molecule. A vector may include sequences that direct autonomous replication, and/or optionally includes sequences that direct integration into host cell DNA. Useful vectors include, for example, plasmids (typically DNA molecules although RNA plasmids are also known), cosmids, and viral vectors. As is well known in the art, the term “viral vector” is widely used refer either to a nucleic acid molecule (e.g., a plasmid) that includes virus-derived nucleic acid elements that typically facilitate transfer of the nucleic acid molecule or integration into the genome (examples include retroviral or lentiviral vectors) or to a viral particle that mediates nucleic acid transfer (examples include retroviruses or lentiviruses). As will be evident to one of ordinary skill in the art, viral particles will typically include various viral components in addition to nucleic acid(s).

### DETAILED DESCRIPTION OF CERTAIN PREFERRED EMBODIMENTS OF THE INVENTION

### I. Overview

Recombinant proteins produced in host cells and/or organisms are assuming an ever-growing role in industrial processes and in medical applications (See, e.g., Walsh, G., Biopharmaceuticals: Biochemistry and Biotechnology, John Wiley & Sons; 2nd ed., New York, 2003). A large number of these proteins, particularly those that are endogenous to eukaryotic species, are glycosylated in their native form. Glycoproteins typically have multiple oligosaccharides attached to them, comprising sugars such as mannose, N-acetyl glucosamine, N-acetyl galactosamine, galactose, fucose and sialic acid. As mentioned above, glycosylation is important to a range of physical, chemical, and biological properties of the glycoprotein. Sialic acid, which typically caps various oligosaccharides, serves a number of important functions. In order to facilitate production of a consistent glycoprotein product by cells in culture, it is desirable that the sialic acid content of the glycoprotein remain relatively constant during over time and over various phases of cell culture. For example, it is desirable to maintain a constant sialic acid content during the growth phase, stationary phase, and death phase of a cell culture.

The instant invention provides RNAi agents targeted to sialidase. The RNAi agents inhibit expression of sialidase when contacted with and/or expressed by cells. Sialidase activity is thereby reduced. The invention further provides cells and cell lines containing any of the RNAi agents. Certain of the cells exhibit reduced sialidase activity relative to cells of the same type that do not contain the RNAi agent. The invention further provides methods of inhibiting sialidase expression and methods of producing a glycoprotein. The glycoprotein has an increased sialic acid content relative to the glycoprotein when produced by comparable cells that do not contain an RNAi agent targeted to sialidase. Surprisingly, it was found that the target of the most effective RNAi agent was selected randomly rather than according to empirical rules and did not conform to predictions of RNAi efficacy. In addition, it was found that stable cell lines that maintain reduced sialidase activity over multiple generations could be produced without loss of cell viability, indicating that cells can tolerate an unexpected degree of sialidase inhibition. Notably and importantly, it was found that glycoproteins produced by such cell lines display an increased sialic acid content, even during the death phase of cell culture.

### II. Design and Synthesis of RNAi Agents

A. Design of RNAi Agents

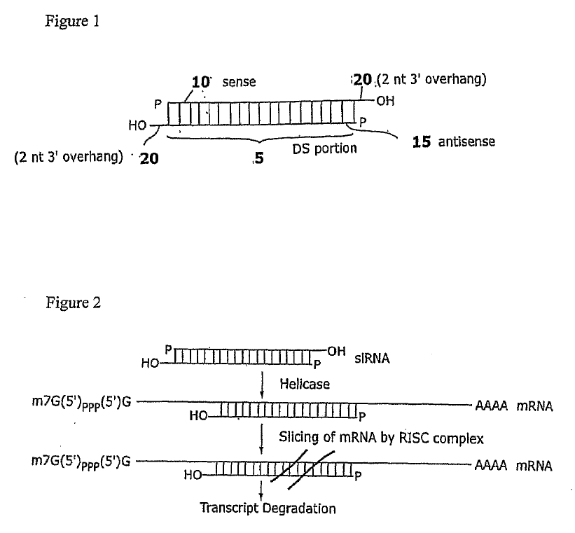

The present invention makes use of RNA interference, which was first described as a phenomenon in which the presence of long dsRNA (typically hundreds of nt) in a cell leads to sequence-specific degradation of mRNA containing a region complementary to one strand of the dsRNA (U.S. Pat. No. 6,506,559). siRNAs were discovered in studies of RNAi in Drosophila, as described in WO 01/75164 and U.S. Pub. Nos. 20020086356 and 20030108923. In particular, it was found that, in Drosophila, long dsRNAs are processed by an RNase III-like enzyme called Dicer (Bernstein et al., Nature 409:363, 2001) into smaller dsRNAs comprised of two 21 nt strands, each of which has a 5′ phosphate group and a 3′ hydroxyl, and includes a 19 nt region precisely complementary with the other strand, so that there is a 19 nt duplex region flanked by 2 nt-3′ overhangs. RNAi was recapitulated in a Drosophila lysate as described in the afore-mentioned publications. FIG. 1 shows a schematic diagram of siRNAs as found in Drosophila. The structure includes a 19 nucleotide double-stranded (DS) portion 5, comprising a sense strand 10 and an antisense strand 15. Each strand has a 2 nt 3′ overhang 20.

Short dsRNAs (siRNAs) having structures such as this act to silence expression of any gene that includes a region complementary to one of the dsRNA strands because a helicase activity unwinds the 19 bp duplex in the siRNA, allowing an alternative duplex to form between one strand of the siRNA (the “antisense” or “guide” strand) and the target transcript. The antisense strand is incorporated into an endonuclease complex referred to as RISC, which is guided to the complementary target RNA. An enzymatic activity present in RISC cleaves (“slices”) at a single location, producing unprotected RNA ends that are promptly degraded by cellular machinery (FIG. 2). Additional mechanisms of silencing mediated by short RNA species (microRNAs) are also known (see, e.g., Ruvkun, G., Science, 294, 797-799, 2001; Zeng, Y., et al., Molecular Cell, 9, 1-20, 2002). The discussion of mechanisms and the figures depicting them are not intended to suggest any limitations on the mechanism of RNA inteference employed in the present invention.

Dicer homologs exist in diverse species ranging from C. elegans to humans (Sharp, Genes Dev. 15; 485, 2001; Zamore, Nat. Struct. Biol. 8:746, 2001), and it has been found that RNAi mechanisms can silence gene expression in a variety of different cell types including mammalian, e.g., human cells. However, long dsRNAs (e.g., dsRNAs having a double-stranded region longer than about 30-50 nucleotides) are known to activate the interferon response in mammalian cells. Thus, rather than achieving the specific gene silencing observed with the Drosophila RNAi mechanism, the presence of long dsRNAs in mammalian cells would be expected to lead to interferon-mediated non-specific suppression of translation, potentially resulting in cell death. Long dsRNAs are therefore not thought to be useful for inhibiting expression of particular genes in mammalian cells.

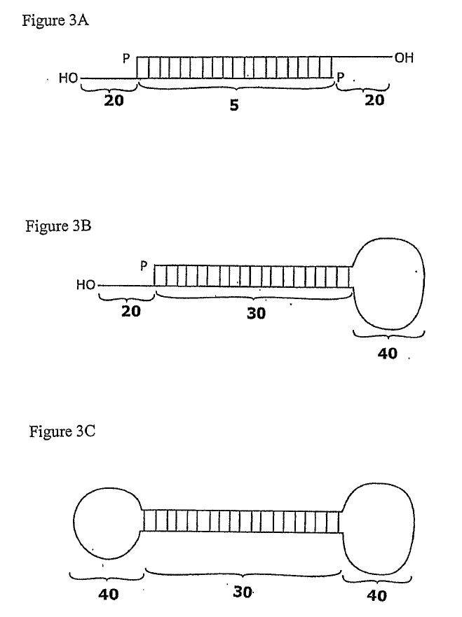

Considerable progress in the practical application of RNAi was achieved with the discovery that siRNAs, when introduced into mammalian cells, can effectively reduce the expression of target genes in a sequence-specific manner via the mechanism described above. For any particular gene target that is selected, the design of RNAi agents for use in accordance with the present invention will preferably follow certain guidelines. Preferred RNAi agents for use in accordance with the present invention include a base-paired region between 15 and approximately 29 nt long, e.g., approximately 19 nt in length, and may optionally have one or more free or looped ends. FIG. 3 presents various structures that can be utilized as an RNAi agent according to the present invention. FIG. 3A shows the structure of a typical siRNA in an exemplary embodiment. FIG. 3B represents an additional structure that may be used to mediate RNA interference. This hairpin structure contains two complementary regions that hybridize to one another to form a duplex region represented as stem 30, a loop 40, and an overhang 20. Such molecules are said to self-hybridize, and a structure of this sort is referred to as an shRNA. shRNAs are processed intracellularly, e.g., by Dicer, to form an siRNA structure such as that shown in FIG. 3A. FIG. 3C shows an alternative structure that could be cleaved to yield an siRNA.

The invention may utilize any structure capable of being processed in vivo to a structure such as that shown in FIG. 3A, so long as the administered agent does not cause undesired or deleterious events such as induction of the interferon response. The invention may also comprise administration of agents that are not processed to precisely the structure depicted in FIG. 3A, so long as administration of such agents reduces sialidase transcript levels or translation. In some cases, an agent that is delivered to or expressed within a cell according to the present invention may undergo one or more processing steps before becoming an active suppressing agent; in such cases, those of ordinary skill in the art will appreciate that the relevant agent will preferably be designed to include sequences that may be necessary for its processing.

In describing RNAi agents and their activities it will frequently be convenient to refer to the agent as having two strands. In general, the sequence of the duplex portion of one strand of the RNAi agent is substantially complementary, e.g., at least 80% complementary, preferably 100% complementary) to the target transcript. The sequence of the duplex portion of the other strand of the RNAi-inducing agent is typically substantially identical to the targeted region of the target transcript, e.g., at least 80% identical, preferably 100% identical. The strand comprising the complementary portion is referred to as the “antisense strand”, while the other strand is often referred to as the “sense strand”.

As mentioned above, certain RNAi agents such as shRNAs contain a single molecule that self-hybridizes to form a hairpin comprising a duplex structure. The duplex structure may be considered to comprise antisense and sense strands, where the antisense strand is a first portion of the molecule that forms or is capable of forming a duplex with a second portion of the molecule and is complementary to the targeted region of the target transcript. The sense strand is the portion of the shRNA molecule which forms or is capable of forming a duplex with the first portion. Typically the strands are substantially complementary, preferably 100% complementary, to each other.

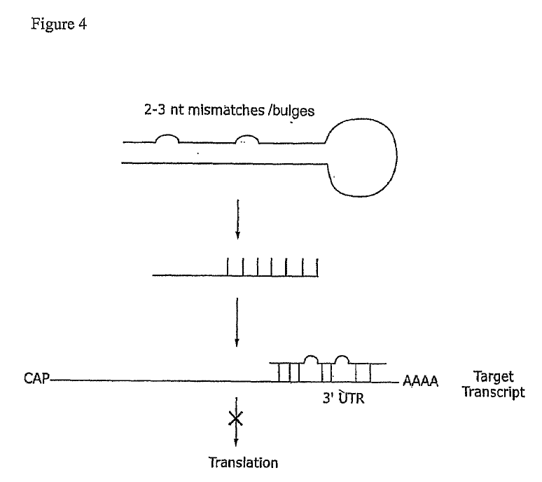

One of ordinary skill in the art will recognize that one or more unpaired nucleotides, e.g., mismatches and bulges, may exist in a duplex formed by an antisense strand and the target. Thus the antisense strand need only be sufficiently complementary to the target such that hybridization can occur, e.g., under physiological conditions in a cell and/or in an in vitro system that supports RNAi, such as the Drosophila lysate system mentioned above. Similarly, one of ordinary skill in the art will recognize that mismatches and bulges may exist in a duplex formed by antisense and sense strands of an RNAi agent such as an siRNA or shRNA. For example, one or both strands may include one or more “extra” nucleotides that form a bulge as shown in FIG. 4 (top).

One of skill in the art will recognize that it may be preferable to avoid mismatches in the central portion of the antisense strand/target RNA duplex (see, e.g., Elbashir et al., EMBO J. 20:6877, 2001). For example, the 3′ and/or 5′ nucleotides of the antisense strand of an siRNA often do not contribute significantly to the specificity of target recognition and may be less critical for target cleavage, while nucleotides in the central region may be more important. One of skill in the art will also recognize that duplex structures interrupted by bulges will typically allow a greater number of unpaired nt than duplex structures in which unpaired nucleotides are present as mismatches.

Some mismatches and/or bulges may be desirable, as duplex formation in the 3′ UTR or elsewhere may inhibit expression of a protein encoded by the transcript by a mechanism related to, but distinct from, the transcript cleavage that occurs in classic RNA inhibition (FIG. 4). For example, certain RNAi agents may act via a translational repression pathway that is used by endogenous RNAs known as microRNAs (miRNAs), which inhibit translation of endogenous mRNAs to which they are partially complementary (Bartel, D P., Cell, 116(2):281-97, 2004; Novina, C. and Sharp, P A, Nature, 430:161-164, 2004; and US Pub. No. 20050059005). A miRNA binds to target mRNA transcripts at partially complementary sites and prevents their translation. RNAi agents having the structure of siRNAs but forming imperfect duplexes with the target transcript can act in a manner similar to miRNAs, i.e., by reducing translation of the transcript rather than decreasing its stability (Doench, J G, et al. Genes & Development, 17:438-442, 2003). It is believed that such siRNAs are processed intracellularly to give rise to single-stranded RNAs that act via the miRNA translational repression pathway.

For the purposes of the present invention, any partly or fully double-stranded short RNA as described herein, one strand of which binds to a target transcript, e.g., a sialidase transcript, and reduces its expression (i.e., reduces the level of the transcript and/or reduces synthesis of the polypeptide encoded by the transcript) is considered to be an RNAi agent, regardless of whether it acts by triggering degradation as is typically the case, inhibiting translation, or by other means. In addition any precursor structure that may be processed in vivo (i.e., within a cell or organism) to generate such an RNAi agent is useful in the present invention.

In general, preferred antisense strands hybridize with a target region in the sialidase transcript that includes exonic sequences. Hybridization with intronic sequences is not excluded, but generally appears not to be preferred in mammalian cells. In certain preferred embodiments of the invention, the antisense strand hybridizes exclusively with exonic sequences. In some embodiments of the invention, the antisense hybridizes with a target region that includes only sequences within a single exon; in other embodiments the target region is created by splicing or other modification of a primary transcript. In other embodiments of the invention the antisense strand hybridizes to a 5′ or 3′ UTR. In general, any site that is available for hybridization with an antisense strand, resulting in slicing and degradation and/or translational repression of the transcript may be utilized in accordance with the present invention. Nonetheless, those of ordinary skill in the art will appreciate that, in some instances, it may be desirable to select particular regions of the sialidase transcript as hybridization targets. For example, it may be desirable to avoid sections of the transcript that may be shared with other transcripts whose degradation is not desired. A database search may be performed to determine whether either strand of an RNAi agent is substantially complementary to any sequence in the genome of a cell or organism into which the agent is to be introduced, and such sequences may be avoided.

Short RNAi agents may be designed according to a variety of approaches. In general, as mentioned above, inventive RNAi agents preferably include a region (the “duplex region”), one strand of which contains an inhibitory region that is sufficiently complementary to a region of the target transcript (the “target region”), so that a hybrid can form in vivo between this strand and the target transcript. In some cases the sequence of an RNAi agent is selected such that the entire antisense strand (including the 3′ overhang if present) is perfectly complementary to the target transcript. However, it is not necessary that overhang(s) are either complementary or identical to the target transcript. Any desired sequence (e.g., UU) may simply be appended to the 3′ ends of antisense and/or sense duplex regions to generate 3′ overhangs. In general, overhangs containing one or more pyrimidines, usually U, T, or dT, are employed. Use of dT rather than T may confer increased stability.

In summary, a short RNAi agent may be designed by selecting a target region and designing the RNAi agent to comprise an antisense strand whose sequence is sufficiently complementary to hybridize to the target, e.g., substantially complementary or 100% complementary to the target transcript over 15-29 nucleotides, e.g., 19 nucleotides, and a sense strand whose sequence is sufficiently complementary to hybridize to the antisense strand, e.g., substantially or, preferably, 100% complementary to the antisense strand. 3′ overhangs such as those described above may then be added to these sequences to generate an siRNA or shRNA structure.

Certain of the RNAi agents of the invention are RNAi expression vectors, which can be used for intracellular (in vivo) synthesis of short RNAi agents such as siRNA or shRNA. Those of ordinary skill in the art will appreciate that if inventive siRNA or shRNA agents are generated in vivo, it is generally preferable that they be produced via transcription of one or more transcription units. The primary transcript may optionally be processed (e.g., by one or more cellular enzymes) in order to generate the final agent that accomplishes gene inhibition. It will further be appreciated that appropriate promoter and/or regulatory elements should be selected to allow expression in mammalian cells. In some embodiments of the invention, it may be desirable to utilize a regulatable promoter; in other embodiments, constitutive expression may be desired.

A variety of nucleic acid constructs can be used to provide templates for transcription of one or more RNAs that hybridize with each other or self-hybridize to form a short RNAi agent. The present invention encompasses constructs encoding one or more siRNA and/or shRNA strands. In addition to a promoter, the construct optionally includes one or more other regulatory elements, e.g., terminator. In certain embodiments of the invention two separate, complementary siRNA strands are transcribed using a single vector containing two promoters, each of which directs transcription of a single siRNA strand, i.e., is operably linked to a template for the siRNA so that transcription occurs. The two promoters may be in the same orientation, in which case each is operably linked to a template for one of the siRNA strands. Alternately, the promoters may be in opposite orientation flanking a single template so that transcription from the promoters results in synthesis of two complementary RNA strands.

In other embodiments of the invention a vector containing a promoter that drives transcription of a single RNA molecule comprising two complementary regions (e.g., an shRNA) is employed. In certain embodiments of the invention a vector containing multiple promoters, each of which drives transcription of a single RNA molecule comprising two complementary regions is used.

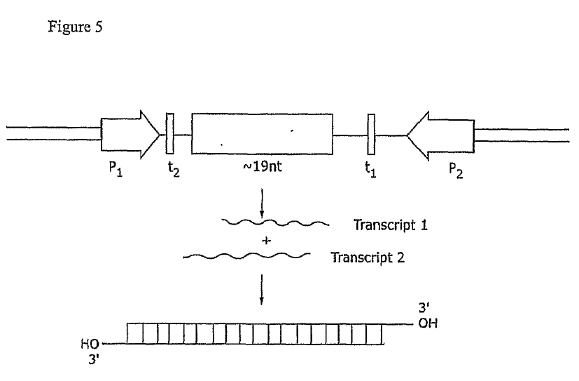

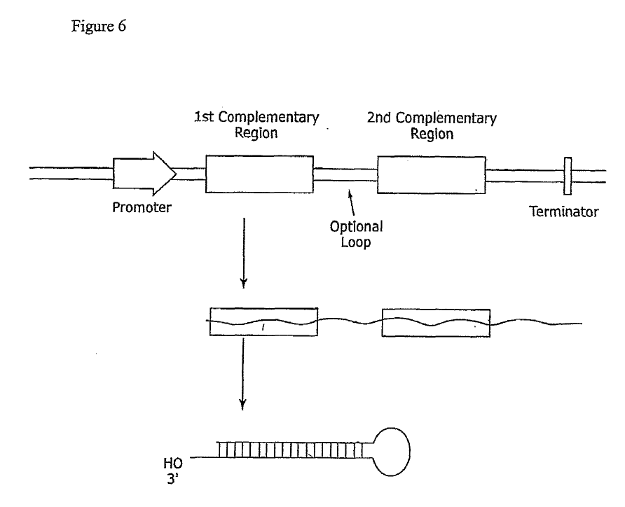

Certain exemplary constructs contain two separate transcribable regions, each of which generates a 21 nt transcript containing a 19 nt region complementary with the other. The length can be varied in accordance with the above descriptions of siRNA and shRNA. Alternatively, a single construct may be utilized that contains opposing promoters P1 and P2 and terminators t1 and t2 positioned so that two different transcripts, each of which is at least partly complementary to the other, are generated (FIG. 5). In another embodiment, an shRNA is generated as a single transcript, e.g., by transcription of a single transcription unit encoding self-complementary regions. FIG. 6 depicts one such embodiment. As indicated, a template is employed that includes first and second complementary regions, and optionally includes a loop region.

In certain preferred embodiments of the invention, the promoter utilized to direct in vivo expression of one or more siRNA or shRNA transcription units is a promoter for RNA polymerase III (Pol III). Pol III directs synthesis of small transcripts that terminate upon encountering a stretch of 4-5 T residues in the template. Certain Pol III promoters such as the U6 or H1 promoters do not require cis-acting regulatory elements (other than the first transcribed nt) within the transcribed region and thus are preferred according to certain embodiments of the invention since they readily permit the selection of desired siRNA sequences. See, e.g., Yu, J., et al., Proc. Natl. Acad. Sci., 99(9), 6047-6052 (2002); Sui, G., et al., Proc. Natl. Acad. Sci., 99(8), 5515-5520 (2002); Paddison, P., et al., Genes and Dev., 16, 948-958 (2002); Brummelkamp, T., et al., Science, 296, 550-553 (2002); Miyagashi, M. and Taira, K., Nat. Biotech., 20, 497-500 (2002); Paul, C., et al., Nat. Biotech., 20, 505-508 (2002); Tuschl, T., et al., Nat. Biotech., 20, 446-448 (2002). Promoters for Pol II may also be used as described, for example, in Xia, H., et al., Nat. Biotechnol., 20, pp. 1006-1010, 2002. As described therein, constructs in which a hairpin sequence is juxtaposed within close proximity to a transcription start site and followed by a polyA cassette, resulting in minimal to no overhangs in the transcribed hairpin, may be employed. In certain embodiments of the invention tissue-specific, cell-specific, or inducible Pol II promoters may be used, provided the foregoing requirements are met. A preferred promoter is a cytomegalovirus (CMV) promoter, optionally including CMV enhancer elements. Other strong PolII promoters (e.g., viral or mammalian promoters such as herpes simplex virus (HSV) promoter, SV40 promoters, retroviral LTR, etc.) may be used. Pol I promoters may also be used in various embodiments (McCown 2003).

A construct that provides a template for synthesis of siRNA or shRNA can be produced using standard recombinant DNA methods and inserted into any of a wide variety of different vectors suitable for introduction into mammalian cells, such as, for example, DNA plasmids or viral vector. See, e.g., Sambrook, supra, and Ausubel, supra. Numerous DNA plasmids are known in the art including those based on pBR322, pUC, etc. It will be appreciated that the vector may already comprise a suitable promoter, in which case it will only be necessary to insert a nucleic acid segment that encodes an inventive short RNAi agent into the vector at an appropriate location with respect to the promoter. Viral vectors that can be used to provide intracellular expression of RNAi agents include, for example, retroviral vectors, lentiviral vectors, adenoviral vectors, adeno-associated virus vectors, herpes virus vectors, etc. For example, see Kobinger, G. P., et al., Nat Biotechnol 19(3):225-30, 2001; Lois, C., et al., Science, 295: 868-872, Feb. 1, 2002, describing the FUGW lentiviral vector; Somia, N., et al. J. Virol. 74(9): 4420-4424, 2000; Miyoshi, H., et al., Science 283: 682-686, 1999; and U.S. Pat. No. 6,013,516.

The present invention encompasses vectors containing siRNA and/or shRNA transcription units, as well as cells containing such vectors or otherwise engineered to contain transcription units encoding one or more siRNA or shRNA strands. The invention therefore provides a variety of viral and nonviral vectors whose presence within a cell results in transcription of one or more RNAs that self-hybridize or hybridize to each other to form an RNAi agent that inhibits expression of sialidase in the cell.

Vectors can be introduced into cells using any of a variety of methods known in the art and described, for example, in Sambrook, supra, and Ausubel, supra. Briefly, such methods include calcium-phosphate transfection, cationic lipid-mediated transfection, electroporation, microparticle bombardment, etc. Cells that have taken up the expression vector are typically selected by growth in or on a selective medium. A stable cell line can be generated as described elsewhere herein. Alternately, transient transfection can be used. In certain preferred embodiments of the invention the vector or a portion thereof containing

B. Synthesis of Short RNAi Agents

Inventive RNAi agents may be prepared according to any available technique including, but not limited to chemical synthesis, enzymatic or chemical cleavage in vivo or in vitro, or template transcription in vivo or in vitro. For example, RNA may be produced enzymatically or by partial/total organic synthesis, and a modified nucleotide can be introduced by in vitro enzymatic or organic synthesis. In one embodiment, an siRNA is prepared chemically. Methods of synthesizing RNA molecules are known in the art, in particular, the chemical synthesis methods as de scribed in Verma and Eckstein, Annu. Rev. Biochem. 67:99-134 (1998). In another embodiment, an siRNA or shRNA is prepared enzymatically. For example, an siRNA or shRNA is prepared by enzymatic processing of a long dsRNA having sufficient complementarity to the desired target RNA. Processing of long dsRNA can be accomplished in vitro, for example, using appropriate cellular lysates and siRNAs or shRNAs can be subsequently purified by gel electrophoresis or gel filtration. For example, RNA can be purified from a mixture by extraction with a solvent or resin, precipitation, electrophoresis, chromatography, or a combination thereof. Alternatively, the RNA may be used with no or minimum purification to avoid losses due to sample processing.

Inventive RNAi agents may be delivered as a single shRNA molecule or as two strands hybridized to one another. For instance, two separate 21 nt RNA strands may be generated, each of which contains a 19 nt region complementary to the other, and the individual strands may be hybridized together to generate a structure such as that depicted in FIG. 3A.

In some embodiments each strand of a short RNAi agent is generated by transcription from a promoter, either in vitro or in vivo. Vectors and constructs suitable for either in vitro or in vivo synthesis of short RNAi agents are described above. In general, the vectors are produced using well known methods employing recombinant DNA technology as described, for example, in Sambrook, supra, and Ausubel, supra. In vitro transcription may be performed using a variety of available systems including the T7, SP6, and T3 promoter/polymerase systems (e.g., those available from Promega, Clontech, New England Biolabs, etc.). When siRNAs are synthesized in vitro they may be allowed to hybridize before contacting them with cells. It will be appreciated that inventive RNAi agent compositions need not consist entirely of double-stranded (hybridized) molecules. For example, such compositions may include a small proportion of single-stranded RNA. Generally, preferred compositions comprise at least approximately 80% dsRNA, at least approximately 90% dsRNA, at least approximately 95% dsRNA, or even at least approximately 99-100% dsRNA. However, the compositions may contain less than 80% hybridized RNA provided that they contain sufficient dsRNA to be effective.

It will be appreciated by those of ordinary skill in the art that agents such as the RNAi agents described herein may be comprised entirely of nucleotides such as those found in naturally occurring nucleic acids, or may instead include one or more analogs of such nucleotides or may otherwise differ from a naturally occurring nucleic acid. Nucleic acids containing modified backbones or non-naturally occurring internucleoside linkages can be used in the present invention. Any nucleic acid structure having the same nucleobase sequence or sequence of nucleobases having approximately equivalent base-pairing specificity as the RNAi agents described herein is encompassed.

Modified nucleic acids need not be uniformly modified along the entire length of the molecule. For example, different nucleotide modifications and/or backbone structures may exist at various positions in the nucleic acid. In certain embodiments of the invention it may be desirable to stabilize the siRNA structure, e.g., by including nucleotide analogs at one or more free strand ends in order to reduce digestion, e.g., by exonucleases. Including deoxynucleotides, e.g., pyrimidines such as deoxythymidines at one or more free ends may serve this purpose. Alternatively or additionally, it may be desirable to include one or more nucleotide analogs in order to increase or reduce stability of the 19 bp stem, in particular as compared with any hybrid that will be formed by interaction of one strand of an RNAi agent with a target transcript. One of ordinary skill in the art will appreciate that the nucleotide analogs may be located at any position(s) where the target-specific activity, e.g., the RNAi mediating activity is not substantially affected, e.g., in a region at the 5′-end and/or the 3′-end of the RNA molecule. For example, in certain embodiments between 1-5 residues at the 5′ and/or 3′ end of an siRNA or shRNA strand is a nucleotide analog. In certain embodiments of the invention one or more of the nucleic acids in an inventive RNAi-inducing agent comprises at least 50% unmodified RNA, at least 80% modified RNA, at least 90% unmodified RNA, or 100% unmodified RNA. In certain embodiments of the invention one or more of the nucleic acids in an inventive RNAi-inducing agent comprises 100% unmodified RNA within the portion that participates in duplex formation in the RNAi-inducing agent.

According to certain embodiments of the invention various nucleotide modifications are used selectively in either the sense or antisense strand of an siRNA, shRNA, or microRNA precursor. For example, it may be preferable to utilize unmodified ribonucleotides in the antisense strand while employing modified ribonucleotides and/or modified or unmodified deoxyribonucleotides at some or all positions in the sense strand. According to certain embodiments of the invention only unmodified ribonucleotides are used in the duplex portion of the antisense and/or the sense strand while the overhang(s) of the antisense and/or sense strand may include modified ribonucleotides and/or deoxyribonucleotides.

Numerous nucleotide analogs and nucleotide modifications are known in the art, and their effect on properties such as hybridization and nuclease resistance has been explored. A number of modifications have been shown to alter one or more aspects of the oligonucleotide such as its ability to hybridize to a complementary nucleic acid, its stability, bioavailability, nuclease resistance, etc. For example, 2′-modifications include halo, alkoxy and allyloxy groups. In some embodiments the 2′-OH group is replaced by a group selected from H, OR, R, halo, SH, SR₁, NH₂, NH_{R}, NR₂ or CN, wherein R is C₁-C₆ alkyl, alkenyl or alkynyl and halo is F, Cl, Br or I. Examples of modified linkages include phosphorothioate and 5′-N-phosphoramidite linkages. U.S. Pat. Nos. 6,403,779; 6,399,754; 6,225,460; 6,127,533; 6,031,086; 6,005,087; 5,977,089, and references therein disclose a wide variety of nucleotide analogs and modifications that may be of use in the practice of the present invention. See also Crooke, S. (ed.) “Antisense Drug Technology: Principles, Strategies, and Applications” (1^{st} ed), Marcel Dekker; ISBN: 0824705661; 1st edition (2001) and references therein. For purposes of the present invention, the chemical elements are identified in accordance with the Periodic Table of the Elements, CAS version, Handbook of Chemistry and Physics, 75th Ed., inside cover, and specific functional groups are generally defined as described therein. Analogs and modifications may be tested using, e.g., the assays described herein or other appropriate assays, in order to select those that effectively reduce expression of viral genes. In certain embodiments the RNAi agent comprises one or more modifications to a sugar, nucleoside, or internucleoside linkage such as any of those described in U.S. Pub. Nos. 20030175950, 20040192626, 20040092470, 20050020525, 20050032733.

It will be appreciated by those of ordinary skill in the art that effective RNAi agents for use in accordance with the present invention may comprise one or more moieties that is/are not nucleotides or nucleotide analogs. In certain embodiments the nucleic acid comprises primarily nucleotide residues but comprises one or more residues that are not nucleotides. For example, in certain embodiments 1, 2, 3, 4, 5, or more of the residues in either strand of an effective silencing agent is not a nucleoside. In certain embodiments the portion of the RNAi agent that participates in duplex formation and/or is complementary to a target transcript consists of nucleosides while the overhang(s) consist of non-nucleoside residues. In certain embodiments of the invention sense and antisense strands of an RNAi agent are attached to one another by a non-nucleoside containing linker.

### III. Sialidase Targets

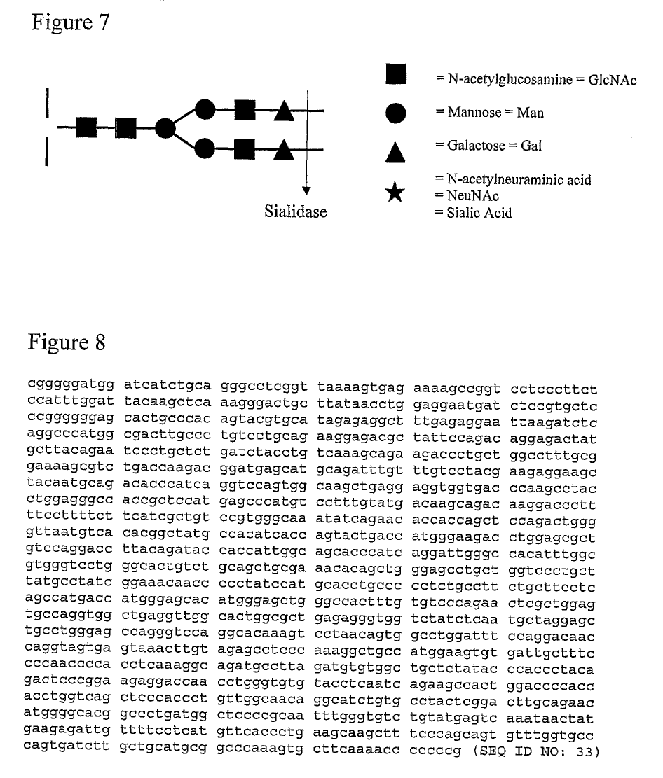

Sialidases, also referred to as exo-α-sialidases, neuraminidases, α-neuraminidases, or acetylneuraminidases, are a group of glycohydrolytic enzymes found in a wide variety of organisms that cleave sialic acid residues from the oligosaccharide components of glycoconjugates, e.g., glycoproteins (FIG. 7). These enzymes are classified under heading EC 3.2.1.18 according to the nomenclature recommendations of the Nomenclature Committee of the International Union of Biochemistry and Molecular Biology. Their systematic name is acetylneuraminyl hydrolase. In general, these enzymes catalyze reactions such as hydrolysis of α-(2→3)-, α-(2→6)-, and α-(2→8)-glycosidic linkages of terminal sialic acid residues in oligosaccharides, glycoproteins, glycolipids, colominic acid and synthetic substrates. For further information see, e.g., Schauer, R. Sialic acids. Adv. Carbolhydr. Chem. Biochem. 40: 131-234, 1982 and Cabezas, J. A. Some questions and suggestions on the type references of the official nomenclature (IUB) for sialidase(s) and endosialidase. Biochem. J. 278: 311-312, 1991.

The invention may be used to inhibit expression of any sialidase. Preferably the sialidase is a eukaryotic sialidase. More preferably the sialidase is a mammalian sialidase, e.g., a rodent (e.g., mouse, rat, or hamster), human, or non-human primate sialidase. Several different sialidases are known to exist in mammals (Monti, E., et al., Genomics, 57: 137-143, 1999, and references therein). Sialidases have been identified in the lysosomal matrix either alone or in association with one or more other proteins. Plasma membrane bound forms of sialidases have been characterized in brain tissue. Cytosolic sialidases have been widely characterized and have been purified from a number of tissues in a number of different species. For example, both the cDNA and the gene encoding cytosolic sialidase from rat skeletal muscle have been cloned (Miyagi 1993). A cDNA encoding a soluble sialidase, originally purified from culture medium of CHO cells, has also been cloned (Ferrari 1994). Several sialidase genes have been identified in humans, of which NEU 2 displays extensive homology with soluble sialidase from rodents (Monti 1999). In various embodiments of the invention cytosolic sialidase, which is present in the cell cytoplasm and is released into culture medium when cells die, is a preferred target for RNAi. In a particularly preferred embodiment the cytosolic sialidase is a hamster sialidase, e.g., a sialidase present in CHO cells.

The following list provides names, gene identifiers, and/or accession numbers for sialidase genes from a variety of species. Any of these genes can be targeted using RNAi in accordance with the present invention. One of ordinary skill in the art will readily be able to identify the relevant sequence in publicly available database, e.g., the Entrez Nucleotides database, which includes GenBank, RefSeq, and PDB and is available, e.g., at the web site having URL www.ncbi.nlm.nih.gov/entrez/query.fcgi?db=Nucleotide. For example, the nucleotide sequence of mRNA encoding hamster cytosolic sialidase is presented in FIG. 8.

Hamster

Cricetulus griseus ATCC CCL61 sialidase mRNA, complete cds
gi|509824|gb|U06143.1|CGUO6143[509824]

Rat

Rat cytosolic sialidase gi|434794
Rat sialisase gi|12862317
Rat sialidase II gi|60552091

House Mouse

Mus musculus neuraminidase 1 (Neu1), mRNA gi|24496769
Mus musculus neuraminidase 2 (Neu2), mRNA gi|7657374
Mus musculus neuraminidase 3 (Neu3), mRNA gi|7710067
Mus musculus neuraminidase 4 (Neu4) mRNA, complete cds gi|32402573

Cow

Bos taurus sialidase 3 (membrane sialidase) (NEU3), mRNA gi|31342798
Bos taurus mRNA for ganglioside sialidase, complete cds gi|4579695

Human

Homo sapiens sialidase 1 (lysosomal sialidase), mRNA (cDNA clone MGC:20282 IMAGE:4101695), complete cds gi|40226388
Homo sapiens sialidase 1 (lysosomal sialidase), mRNA (cDNA clone MGC:1553 IMAGE:3506824), complete cds gi|33875782
Homo sapiens sialidase 1 (lysosomal sialidase) mRNA, complete cds gi|30583250
Homo sapiens lysosomal sialidase mRNA, complete cds gi|4099140
Homo sapiens sialidase 2 (cytosolic sialidase), mRNA (cDNA clone MGC:95436 IMAGE:7217011), complete cds gi|46575655
Homo sapiens mRNA for ganglioside sialidase, complete cds gi|5731569
Homo sapiens sialidase 4, mRNA (cDNA clone IMAGE:4156395), complete cds gi|15991858

### IV. Cells and Cell Lines

Any eukaryotic cells can be used in the practice of the present invention. Appropriate host cells include any of those routinely used in expressing eukaryotic or mammalian polypeptides, including, for example, fungi, such as yeast (e.g., Pichia pastoris); insect cells (e.g., Sf9), plant cells, and animal cells, e.g., mammalian cells such as mouse, hamster, non-human primate, or human cells. Preferably the cells are mammalian cells. Specific examples include Chinese hamster ovary (CHO), R1.1, B-W, L-M, COS-1, COS-7, BSC-1, BSC-40, BMT-10, BHK, HeLa, HEK-293, NIH/3T3, HT1080, 293T, WI-38, and CV-1. For an extensive list of mammalian cell lines, those of ordinary skill in the art may refer to the American Type Culture Collection catalog (ATCC®, Manassas, Va.). In general, the cells may be of any cell type, e.g., fibroblast, myoblast, T or B cell, macrophage, epithelial cells, etc. Typically the cells express one or more enzymes that glycosylate the polypeptide of interest. In preferred embodiments of the invention the cells are of a type that is widely used for production of glycoproteins. For example, the cell can be a myeloma cell, a hybridoma cell, or a CHO cell. In many cases a number of different subclones of any particular cell line are known in the art. For example, the CHO-K1 cell line (ATCC Number CRL 9618™) is a subclone of the parental CHO cell line, which was derived from the ovary of an adult Chinese hamster (Puck et al., J. Exp. Med. 108: 945, 1958. Numerous CHO cell lines, some of which produce a glycoprotein of interest, are available from the ATCC, e.g., CTLA4 Ig-24 (ATCC Number CRL-10762™), which produces the fusion protein CTLA4Ig; CHO-ICAM-1 (ATCC Number CRL-2093™), which produces intracellular cell adhesion molecule-1 (ICAM-1), etc.

The cell lines of the invention in general comprise a plurality of any of the cells of the invention or descendents thereof that can be maintained continuously in culture over an extended period of time, typically months or years, e.g., the cells are immortalized. Thus cells of any particular cell line are generally of the same cell type and will typically comprise the same nucleic acid that encodes a selectable marker and the same template(s) for transcription of an RNAi agent that reduces expression of sialidase. In certain embodiments of the invention a cell line is derived from a single cell, e.g., by a single step cloning procedure, resulting in a clonal cell line. While cell lines that contain a heterogenous population of cells not derived from a single cell are not excluded, cell lines derived from single cells are generally preferred since, for example, they will typically display more uniform extents of RNAi-mediated silencing and/or production of a polypeptide of interest. Methods for generating clonal cell lines are well known in the art. For example, cells can be transformed with an RNAi expression vector comprising a selectable marker (e.g., a drug resistance gene), and cells that have taken up and express the marker can be selected. Single cells, or colonies derived from single cells, are identified and expanded in culture. Optionally, cells can be plated at a low density to facilitate the isolation of single cells or colonies derived from single cells.

In certain embodiments of the invention an RNAi expression vector is introduced into the cells. The cells may or may not express a glycoprotein of interest prior to introduction of the RNAi expression vector. For example, in certain embodiments an RNAi expression vector targeted to sialidase is introduced into cells that do not express a polypeptide of interest, e.g., a heterologous polypeptide. The cells or cell line may be tested to confirm reduced sialidase activity. A stable cell line is optionally derived from such cells. Such cells or cell lines can be used as a “universal host” for expression of any polypeptide of interest. The cells or cell line may then be transformed with an expression vector that encodes any polypeptide of interest, e.g., a polypeptide that is glycosylated by the cell. The cells or cell line may be tested to confirm expression of the polypeptide. A stable cell line that expresses the polypeptide and exhibits reduced sialidase activity is optionally derived from the cells or cell line. Alternately, if the cell is a myeloma cell, the myeloma cell may be fused with a plasma cell that produces an antibody of interest, e.g., to generate a hybridoma.

Alternately, an RNAi expression vector may be introduced into cells or a cell line that already express a polypeptide of interest, e.g., a polypeptide that is glycosylated by the cell. The cells or cell line may be tested to confirm that it exhibits reduced sialidase activity. The cells or cell line may also be tested to confirm that it still expresses the polypeptide of interest. A stable cell line that expresses the polypeptide and exhibits reduced sialidase activity is optionally derived from the cells or cell line. Preferably viability of the cells is not adversely affected by presence of the RNAi agent. For example, preferably the level of viability and/or growth rate of a cell that expresses an RNAi agent is between 75% and 100% of the level of viability and/or growth rate of a comparable cell that does not express the RNAi agent.

### V. Glycoprotein Production

The cells and methods of the invention may be used to produce any polypeptide of interest, e.g., any polypeptide that is glycosylated in mammalian cells. The polypeptide may belong to any of a large number of different protein families. Preferably the polypeptide is a mammalian glycoprotein. In certain embodiments the polypeptide is a therapeutic glycoprotein, i.e., a glycoprotein that is administered to an organism, preferably a human, to treat or prevent a disease or clinical condition. The polypeptide can be heterologous or non-heterologous to the cell.

Exemplary polypeptides of interest include hormones (e.g., insulin, thyroid hormone, catecholamines, gonadotrophines, trophic hormones, prolactin, oxytocin, dopamine, bovine somatotropin, leptins and the like), growth hormones (e.g., human growth hormone), growth factors (e.g., epidermal growth factor, nerve growth factor, insulin-like growth factor and the like), growth factor receptors, cytokines and immune system proteins (e.g., interleukins, colony stimulating factor (CSF), granulocyte colony stimulating factor (G-CSF), granulocyte-macrophage colony stimulating factor (GM-CSF), tumor necrosis factor (TNF), interferons such as IFNα, IFNβ, or IFNγ, erythropoietin, integrins, addressins, selectins, homing receptors, T cell receptors, immunoglobulins, soluble major histocompatibility complex antigens, immunologically active antigens such as bacterial, parasitic, or viral antigens or allergens or autoantigens, enzymes (e.g., tissue plasminogen activator, streptokinase, cholesterol biosynthestic or degradative enzymes, steroidogenic enzymes, kinases, phosphodiesterases, methylases, de-methylases, dehydrogenases, cellulases, proteases, lipases, phospholipases, aromatases, cytochromes, adenylate or guanylaste cyclases, neuramidases and the like), receptors (steroid hormone receptors, peptide receptors), binding proteins (sterpod binding proteins, growth hormone or growth factor binding proteins and the like), transcription and translation factors, oncoproteins or proto-oncoproteins (e.g., cell cycle proteins), muscle proteins (myosin or tropomyosin and the like), myeloproteins, neuroactive proteins, tumor growth suppressing proteins (angiostatin or endostatin, both which inhibit angiogenesis), anti-sepsis proteins (bectericidal permeability-increasing protein), structural proteins (such as collagen, fibroin, fibrinogen, elastin, tubulin, actin, and myosin), blood proteins (thrombin, serum albumin, Factor VII, Factor VIII, insulin, Factor IX, Factor X, tissue plasminogen activator, Protein C, von Willebrand factor, antithrombin III, glucocerebrosidase, granulocyte colony stimulating factor (GCSF) or modified Factor VIII, anticoagulants such as huridin and the like).

In a preferred embodiment the glycoprotein is an antibody, e.g., a monoclonal antibody. Monoclonal antibodies are assuming an ever more important role in medicine, for both diagnostic and therapeutic purposes in diseases ranging from rheumatoid arthritis to cancer. For example, Herceptin® (trastuzamab) is a monoclonal antibody that binds to a protein known as HER2 and is used to treat breast cancer. Rituxan® (rituximab), Humira® (adalimamab), Remicade® (infliximab) are other monoclonal antibodies currently approved for treatment of various diseases.

The protein can be a fusion protein, e.g., it can contain a first domain from a first polypeptide of interest and a second domain from a second polypeptide of interest, wherein the two domains are fused together, optionally separated by one or more additional domains. For example, the protein can comprise an immunoglobulin domain or portion thereof fused to a non-immunoglobulin polypeptide. In some embodiments the protein comprises one or more portions of an antibody molecule, e.g., an Fc domain. Of course one or more portions of the polypeptide may have a sequence that does not naturally occur in any known polypeptide. The polypeptide may be an altered form of a naturally occurring polypeptide, wherein the alteration comes about as a result of deliberate mutation of a coding sequence for a known polypeptide using recombinant DNA technology (e.g., as in the case of Aranesp®, an engineered form of erythropoietin), or the sequence may be invented in part or entirely by man. In some embodiments the protein comprises a “tag” that can be used, e.g., for purposes of detecting and/or purifying the protein. The tag can be, for example, an epitope tag such as an HA tag, Myc tag, FLAG tag, maltose binding protein tag, GST tag, or the like. The protein can comprise a cleavage site such as a specific site for cleavage by a protease, e.g., so that a tag can subsequently be easily removed.

In order to produce a glycosylated polypeptide using the inventive methods, a host cell that expresses the polypeptide is needed. A large number of cell lines that produce any of a wide variety of recombinant proteins are known in the art. Any such cell line can be used. Alternately, a host cell that expresses a polypeptide of interest can be generated by introducing an expression vector comprising a coding sequence for the polypeptide into a suitable host cell using any of a large number of art-recognized methods for accomplishing nucleic acid transfer, a number of which are mentioned above. As mentioned above, in some embodiments of the invention the expression vector encoding the polypeptide is introduced into a host cell that expresses an RNAi agent targeted to sialidase while in other embodiments the expression vector is introduced into a host cell that does not express an RNAi agent targeted to sialidase. Of course cells such as myeloma cells, hybridoma cells, etc., that produce a glycoprotein of interest, e.g., a monoclonal antibody, without having an expression vector introduced into them can also be used.

In some embodiments, e.g., if the cells do not express an RNAi agent targeted to sialidase, the cells are maintained in culture in the presence of an RNAi agent, e.g., an siRNA, which is targeted to sialidase. In other embodiments an RNAi expression vector is introduced into the cells, so that they will express an RNAi agent such as an shRNA targeted to sialidase. Optionally a cell line that stably expresses the RNAi agent is generated. In either case, the cells are maintained in culture under suitable conditions for expression of the polypeptide. For example, if expression of the polypeptide and/or expression of an RNAi agent is under control of an inducible promoter, a suitable inducing agent may be added to the culture.

The invention can be practiced on a laboratory or research scale for production of relatively small amounts of glycoprotein, e.g., milligrams to grams. However, in certain embodiments of the invention larger scale cultures are used. The cells may be maintained in a large bioreactor for production of gram to kilogram quantities of the glycoprotein. The culture may be, e.g., a batch culture, fed batch culture, semi-fed batch, or continuous culture. After a period of time cells and/or tissue culture medium is harvested, and protein is purified therefrom. In accordance with the terminology as commonly accepted in the art and described, e.g., in Stephanopolous, G., ed. Bioprocessing. Second ed. Biotechnology, ed. H.-J. Rehm, et al. Vol. 3. 1993, VCH Publishers Inc.: New York, cell culture strategies can be classified into one of three general modes, i.e., batch or fed-batch operations, the semi-continuous or cut-and-feed strategy (which may also be referred to as semi-batch), and perfusion culture. Batch culture is usually performed using suspension culture cells in a stirred tank bioreactor, although in the case of a microreactor as described herein, stirring may or may not be performed. Product is harvested from the medium at the end of the batch cycle. Fed-batch culture differs from batch culture in that nutrients (or solutions of interest such as reactants, buffers, etc.) are added either continuously or periodically during the batch cycle. The semi-continuous or cut-and-feed strategy also typically employs stirred tank, homogeneously mixed bioreactors. In this operating strategy a bioreactor is inoculated with cells, which are then allowed to grow for a period of time, often until the culture is approaching early stationary phase. A large fraction of the cell culture broth is then harvested, usually on the order of 70-90%, and the bioreactor replenished with fresh medium. The cycle is then repeated. Perfusion operations retain cells within the reactor while allowing a cell-free sidestream to be removed; they can be subdivided into two categories, the homogeneous systems such as the perfusion chemostat or heterogeneous systems like hollow fiber or fluidized bed bioreactors. It is to be understood that these descriptions are merely representative and are not intended to limit the invention or its modes of operation in any way and that they can be modified as appropriate in the context of any particular glycoprotein of interest.

The polypeptide of interest is purified from the cells and/or medium using any suitable method of which many are known in the art and include, for example, ion-exchange chromatography, affinity chromatography (e.g., immunoaffinity chromatography), hydrophobic interaction chromatography, size-exclusion chromatography, precipitation, dialysis, and combinations thereof, etc. See, e.g., Roe, S., (ed.), Protein Purification Techniques: A Practical Approach, Oxford University Press; 2nd edition, 2001; Hardin, C., et al. (eds.), Cloning, Gene Expression and Protein Purification: Experimental Procedures and Process Rationale, Oxford University Press, 2001; Scopes, R., Protein Purification: Principles and Practice, Springer; 3rd ed., 1993, etc. The skilled artisan will be able to select methods appropriate for any particular glycoprotein of interest.

### VI. Extent of Sialidase Inhibition and Methods of Testing

The RNAi agents of the invention preferably inhibit sialidase expression, e.g., mRNA and/or protein expression. In a preferred embodiment both mRNA and protein expression are reduced. Preferably the reduction in sialidase expression results in a detectable decrease in sialidase activity. In preferred embodiments an RNAi agent reduces sialidase mRNA levels by at least 2-fold, preferably at least 5-fold, more preferably between 10 and 100-fold, yet more preferably between 100 and 1000-fold, and yet more preferably between 1000-fold and 10,000-fold, or to an even greater extent, e.g., between 10,000 and 20,000-fold, between 20,000 and 30,000-fold, etc., when cells are contacted with the RNAi agent and/or when the RNAi agent is expressed by the cells. Preferably the amount of sialidase protein in the cells is reduced by at least 2-fold, preferably at least 5-fold, more preferably between 10 and 100-fold, yet more preferably between 100 and 1000-fold, in cells that are contacted with the RNAi agent and/or when the RNAi agent is expressed by the cells. Preferably the decrease in sialidase expression results in a decrease in sialidase activity in the cells (e.g., in a lysate obtained from the cells), in tissue culture medium in which the cells are cultured, or, preferably in both. For example, the sialidase activity may be decreased by between 20% and 50%, between 50% and 60%, between 60% and 70%, between 70% and 80%, between 80% and 90%, or by between 90% and 100% of its value in the absence of the RNAi agent. The decrease can be measured after a predetermined culture interval (e.g., 24, 48, 72, 144, 216 hours or more), and/or one or more culture phases.

It may be desirable to assess the ability of an RNAi agent to inhibit sialidase expression and/or to assess the effects of such inhibition on sialidase activity and/or to assess the effect of such inhibition on the sialic acid content of a glycoprotein produced by a cell that contains an inventive RNAi agent targeted to sialidase.

In general, a wide variety of methods can be used to measure sialidase mRNA level (e.g., Northern blots, RT-PCR, microarray analysis) or protein level (e.g., Western blot, immunoassay, etc.). Assays of sialidase activity are a preferred method. Any suitable assay for sialidase activity known in the art can be used. For example, a thiobarbituric acid assay (TAA) is widely used to measure the amount of total sialic acid per protein (Hammond and Papermaster, 1976). Briefly, the TAA assay measures the sialic acid content of a purified protein. The assay involves cleaving sialic acid off one or more glycoproteins and quantifying the released sialic acid, e.g., using a spectrophotometer (Gramer, M J and Goochee, C F., Glycosidase activities in CHO cell lysate and cell culture supernatant. Biotechnolology Progress 9:366-373, 1993). The sialidase activity could be measured on a preparation comprising multiple different glycoproteins (e.g., a cell lysate, tissue culture medium), e.g., to obtain an overall measure of sialidase activity, or one or more glycoproteins could be partly or fully purified. Generally the value is compared with a glycoprotein preparation obtained from similar cells that are not contacted with and do not express an RNAi agent targeted to sialidase. A control cell may be contacted with or may express an RNAi agent that is not targeted to sialidase, e.g., to ensure that any effect on sialidase activity is specific and is not simply an effect of the presence of the RNAi agent in the cell.

To test whether a reduction in sialidase activity improves the sialic acid content of a glycoprotein of interest, the glycoprotein should be sufficiently purified, e.g., from cell lysate and/or culture medium, so that the analysis is performed only on the glycoprotein of interest. The glycoprotein is then subjected to an assay such as a TAA assay to quantitate the sialic acid content. MALDI/TOF mass spectrometry and/or High Performance Anionic Exchange Chromatography (HPAEC) combination may be used to obtain the actual structure of glycoprotein. These methods may be used, e.g., to confirm results of a TAA assay.

A wide variety of additional or alternative assays could be performed. For example, the stability of a glycoprotein produced using the inventive methods (e.g., in a cell that expresses an RNAi agent targeted to sialidase) can be compared with the stability of the same glycoprotein when produced in cells that do not express an RNAi agent targeted to sialidase. Stability could be measured in vitro or the glycoprotein could be administered to an animal and the clearance in the body could be measured. The glycoprotein could be labeled to facilitate such analysis. See, e.g., Flesher, A R, Marzowski, J, Wang, W C, Raff, W V. 1995. Fluorophore-labeled carbohydrate analysis of immunoglobulin fusion proteins: correlation of oligosaccharide content with in vivo clearance profile. Biotech Bioeng 46: 399-407. Glycan site occupancy can also be measured as described, e.g., in the Examples.

Various bioassays could also be performed, wherein the bioassay measures a biologically relevant activity of the glycoprotein that is indicative of its sialic acid content and/or stability, e.g., a glycoprotein with a higher sialic acid content will exhibit greater activity in the assay. For example, cell proliferation assays, chemotactic assays, etc., could be performed, or the therapeutic effect of a glycoprotein could be measured after administration to an organism.

### VII. Kits

The invention provides kits comprising one or more RNAi agents targeted to sialidase. The RNAi agent may be any siRNA, shRNA, or RNAi expression vector as described herein. The kits may further comprise at least one item selected from the group consisting of: (a) a cell; (b) a transfection reagent; (c) culture medium; (d) a selection agent; (e) an expression vector for insertion of a heterologous protein coding sequence; and (f) instructions for use. The invention further provides kits comprising one or more of the cells or cell lines of the invention. The kits may further comprise at least one item selected from the group consisting of: (a) a transfection reagent; (b) culture medium; (c) a selection agent; (d) an expression vector for insertion of a heterologous protein coding sequence; and (e) instructions for use.

Kits will generally include one or more vessels or containers so that certain of the individual reagents may be separately housed. The kits may also include a means for enclosing the individual containers in relatively close confinement for commercial sale, e.g., a plastic box, in which instructions, packaging materials such as styrofoam, etc., may be enclosed.

### VIII. Transgenic Animals

The present invention encompasses transgenic non-human animals engineered to contain or express an inventive RNAi agent. Such animals are useful for studying the function and/or activity of inventive RNAi agents, and/or for studying the function of sialidase, e.g., its role in development, etc. In addition, the transgenic animals are sources of cells with reduced sialidase activity. The cells may be used, e.g., for production of glycoproteins as described herein. As used herein, a “transgenic animal” is a non-human animal in which one or more of the cells of the animal, preferably most or all of the cells, includes a transgene. A transgene is exogenous DNA or a rearrangement, e.g., a deletion of endogenous chromosomal DNA, which preferably is integrated into or occurs in the genome of the cells of a transgenic animal. Preferably the transgene comprises a promoter operably linked to a nucleic acid such that expression of the nucleic acid occurs in the cell.

A transgene can direct the expression of an RNAi agent in one or more cell types or tissues of the transgenic animal. Certain preferred transgenic animals are non-human mammals, e.g., rodents such as rats, mice, or hamsters. Other examples of transgenic animals include non-human primates, sheep, dogs, cows, goats, amphibians, and the like. The RNAi agent may be, for example, an siRNA or shRNA. The RNAi-inducing agent can be targeted to any potential sialidase target portion, e.g., a target portion listed in any of Tables 1A, 1B, 17, 18, 20, and/or 34. In certain embodiments the RNAi agent is targeted to a target region whose sequence is selected from SEQ ID NOs: For example, in preferred embodiments the RNAi agent has an antisense strand that is complementary to any of the foregoing target regions and a sense strand that forms a duplex with the antisense strand.

Methods for making transgenic non-human animals are known in the art. Briefly, these methods include (i) introducing an appropriate vector comprising the transgene into nuclei of fertilized eggs by microinjection, followed by transfer of the egg into the genital tract of a pseudopregnant female; and (ii) introducing an appropriate vector comprising a transgene into a cultured somatic cell (e.g., using any convenient technique such as transfection, electroporation, etc.), selecting cells in which the transgene has integrated into genomic DNA, transferring the nucleus from a selected cell into an oocyte or zygote, optionally culturing the oocyte or zygote in vitro to the morula or blastula stage, and transferring the embryo into a recipient female. According to other methods, a retroviral vector comprising the transgene is used. The retroviral vector is introduced into cells either as DNA plasmid or as a viral particle, by infection. Cytoplasmic microinjection of an appropriate vector into an oocyte or embryonic cell can also be used. Sperm-mediated transgenesis is also encompassed. Heterozygous or chimeric animals obtained using these methods are identified and bred to produce homozygotes.

The vector is preferably an RNAi expression vector targeted to sialidase. The vector comprises a promoter operably linked to a template for transcription of a short RNAi agent. The RNAi agent may be produced as a single RNA molecule comprising complementary portions or as two RNA molecules that hybridize within the cell, as described above. The promoter may, but need not be, derived from the species of the transgenic animal. RNA Pol I, II, or III promoters can be used. The promoter can be constitutive or inducible.

In a preferred embodiment the transgenic animals are rodents, e.g., mice or rats. Mice and rats that express RNAi-inducing agents have been produced using a variety of different approaches (see, e.g., Hasuwa, et al, FEBS Lett. 2002 Dec. 4; 532(1-2):227-30. Xia, et al., Nat. Biotechnol., 20(10):1006-10, 2002; Rubinson, et al, Nat. Genet., 33(3):401-6, 2003).

### EXEMPLIFICATION

### Example 1

### Design of siRNAs to Inhibit Sialidase

Cytosolic sialidase cDNA in CHO cells (FIG. 8; SEQ ID NO: 33) consists of 1366 nucleotides and therefore contains 1348 possible potential 19 nucleotide targets for RNAi, i.e., 1348 antisense strands containing a 19 nt inhibitory region perfectly complementary to the sialidase mRNA transcript could potentially interfere with sialidase expression.

Five siRNAs targeted to different regions of the sialidase cDNA were designed based on empirical rules (Elbashir et al, 2001c, 2002). The siRNAs were designed to contain 21-nt sense and 21-nt antisense strands paired in a manner to have 2-nt 3′ overhangs on each strand. Target regions at least 50-100 nt downstream of start codon were selected. 5′ or 3′ untranslated (UTR) regions were avoided. Based on the cDNA sequence, 23-nt sequence motifs of the form 5′-AA(N₁₉)TT-3′ or 5′-NN(N₁₉)TT-3′, where N is any nucleotide were identified. Regions selected for siRNA design had a 30%-70% G/C ratio. In those cases where the targeted region was of the form 5′-AA(N₁₉)TT-3′, the antisense siRNA stands were designed as the perfect RNA complement to position 1 to 21 of the 23 nt motif, and the sense strand was designed as the RNA form of positions 3-23 of the 23 nt motif. In those cases where the targeted region was of the form 5′-NN(N₁₉)TT-3′, the antisense siRNA strand was the perfect RNA complement to N₁₉, and two U residues were added at the 3′ end. The sense strand was designed as the RNA form of positions 3-23 of the 23 nt motif. Both strands thus included 2 U residues at the 3′ end so that both strands of the siRNA duplex contained 2 nt 3′ overhangs consisting of UU. Rather than selecting U, any other nucleotide (or no nucleotide) could have been selected. For example, T or dT could have been used.

In addition to selecting 5 siRNAs using the empirical rules, 5 siRNAs targeted to randomly selected 19 nt (N₁₉) regions of the sialidase cDNA were also selected. The antisense strands of the siRNAs were designed as the perfect RNA complement to the N₁₉ region while the sense strands were identical to the N₁₉ region in RNA form. Both strands also included 2 U residues at the 3′ end so that both strands of the siRNA duplex contained 2 nt 3′ overhangs consisting of UU. Rather than selecting U, any other nucleotide (or no nucleotide) could have been selected. For example, T or dT could have been used.

In all cases a BLAST search was performed to ensure that the siRNA strands displayed no substantial homology to genes other than cytosolic sialidase.

Table 1 shows the sequences of the 10 target regions and corresponding siRNA antisense and sense strands. In each box in the column labeled “Target Sequence”, the target region sequence occupies positions 1-19 (i.e., N1-N19) of the upper sequence. The upper sequence is the siRNA sense strand sequence, and the lower sequence is the antisense strand sequence. Positions 1-19 of the lower sequences, in the 5′ to 3′ direction are perfectly complementary to the target region. siRNAs S1, S4, S8, S9, and S10 were designed to target randomly selected target regions. siRNAs S2, S3, S5, S6, and S7 were designed to target regions that were selected according to the empirical rules.

TABLE 1
Sialidase siRNA Sequences

| | | |
|---|---|---|
| | | SEQ ID |
| Description | Target Sequence | NO: |

| | | |
|---|---|---|
| Sialidase | 5′ A U C A U C U G C A G G G C C U C G G U U 3′ | 1 |
| siRNA | 3′ U U U A G U A G A C G U C C C G G A G C C 5′ | 2 |
| Sequence S1 | | |
| Sialidase | 5′ G G C U G C C A U G G A A G U G U G A U U 3′ | 3 |
| siRNA | 3′ U U C C G A C G G U A C C U U C A C A C U 5′ | 4 |
| Sequence S2 | | |
| Sialidase | 5′ G C A G A A G A C C C U G C U G G C C U U 3′ | 5 |
| siRNA | 3′ U U C G U C U U C U G G G A C G A C C G G 5′ | 6 |
| Sequence S3 | | |
| Sialidase | 5′ G G A G A C U A U G C U U A C A G A A U U 3′ | 7 |
| siRNA | 3′ U U C C U C U G A U A C G A A U G U C U U 5′ | 8 |
| Sequence S4 | | |
| Sialidase | 5′ G C C G G U C C U C C C U U C U C C A U U 3′ | 9 |
| siRNA | 3′ U U C G G C C A G G A G G G A A G A G G U 5′ | 10 |
| Sequence S5 | | |
| Sialidase | 5′ G A C G G A U G A G C A U G C A G A U U U 3′ | 11 |
| siRNA | 3′ U U C U G C C U A C U C G U A C G U C U A 5′ | 12 |
| Sequence S6 | | |
| Sialidase | 5′ G C A A G C U U U C C C A G C A G U G U U 3′ | 13 |
| siRNA | 3′ U U C G U U C G A A A G G G U C G U C A C 5′ | 14 |
| Sequence S7 | | |
| Sialidase | 5′ G A C U A U G C U U A C A G A A U C C U U 3′ | 15 |
| siRNA | 3′ U U C U G A U A C G A A U G U C U U A G G 5′ | 16 |
| Sequence S8 | | |
| Sialidase | 5′ G A U U U G U U U G U C C U A C G A A U U 3′ | 17 |
| siRNA | 3′ U U C U A A A C A A A C A G G A U G C U U 5′ | 18 |
| Sequence S9 | | |
| Sialidase | 5′ C U C G G A C U U G C A G A A C A U G U U 3′ | 19 |
| siRNA | 3′ U U G A G C C U G A A C G U C U U G U A C 5′ | 20 |
| Sequence S10 | | |

### Example 2

### siRNAs Targeted to Sialidase Reduce Sialidase mRNA Expression

Materials and Methods

CHO Cell Culture

Recombinant human IFN-γ was produced by a CHO cell line (originally provided by Dr. Walter Fiers, University of Ghent, Belgium) cotransfected with genes for dihydrofolate reductase (DHFR) Cells were cultured in Dulbecco's minimum essential medium (DMEM) and selected for growth in the presence of 2.5×10⁻⁷ M methotrexate (Sigma, St Louis, Mo.), 20 units ml⁻¹ penicillin-20 □g ml⁻¹ streptomycin mix (Invitrogen) and 10% Heat-Inactivated fetal bovine serum (Invitrogen). Attached cell lines were grown in 6-well-plates, T-75 flasks, and T-150 flasks by inoculating about 1×10⁵/mL cells. Transformation from attached cell lines to suspension cell lines was done by switching the medium from DMEM to HYQPF-CHO (HyClone) supplemented with 4 mM Glutamine and 0.1% Pluronic (Invitrogen). Serum content was gradually reduced from 10% to 0%. Cell density and viability were determined with a Neubauer Hemacytometer (Reichert, Buffalo, N.Y.). Prior to cell counting, 0.4% trypan blue solution (Sigma) was used to dilute the sample with an equal volume. Trypsin EDTA (Invitrogen) was often added to prevent cell attachment and aggregation before counting.

siRNA Transfection, Cell Collection, and RNA Extraction

siRNA designed as described in Example 1 were obtained from Dharmacon (Lafayette, Colo.). The siRNAs were deprotected and annealed according to the manufacturer's directions. CHO cells were inoculated at a concentration of 1×10⁵/well in a 6-well plate 24 hours prior to transfection. On the day of transfection, 5 nmol of siRNA duplex was diluted to 50 □L in OptiMEM (Invitrogen) and 10 □L of Lipofectamine 2000 was diluted to 50 □L in OptiMEM in separate tubes. After 5 minutes incubation at room temperature, diluted siRNA was mixed gently with the Lipofectamine 2000 mixture and incubated for 20 minutes at room temperature. During the incubation period, CHO cell medium was aspirated and the cells were washed twice with PBS after which 900 □L of serum free OptiMEM was added. 100 □L of siRNA solution was subsequently added to each well. After 4 hours of incubation at 37° C., each well was washed with PBS and fresh DMEM (supplemented with 20% IFS) was introduced. Every 24 hours, a cell count was performed, and cells were collected after incubation with 0.05% Trypsin/EDTA solution for 5 minutes at 37° C. 1×10⁶ cells were then washed twice with PBS before storing at −20° C. for future sialidase assays. RNA was extracted from an additional 1×10⁶ isolated cells using a Qiagen RNeasy Mini Kit. RNAse free DNase (Qiagen) treatment was performed to remove the residual DNA present in the RNA prep.

RT-PCR Assay of Sialidase mRNA Expression

Reagents for the RT-PCR assay were obtained from Invitrogen (Carlsbad, Calif.). 1 □g of RNA was thawed on ice and immediately mixed with 1 □L of 50 □M oligo(dT)₂₀ and 10 □M of dNTP mix. DEPC-treated water was then added to a final volume of 10 □L and the whole mixture was incubated at 65° C. for 5 minutes followed by brief incubation on ice. 10 □L of cDNA synthesis mix (which consisted of 2 □L 10×RT buffer, 4 □L 25 mM MgCl₂, 2 □L 0.1M DTT, 1 □L 40 U/□L RNaseOUT, and 1 □L 200 U/□L SuperScript III RT) was added to the initial mixture. The final solution was incubated at 50° C. for 1 hour and 85° C. for 7 minutes. 1 □L RNaseH was added, and the solution was incubated at 37° C. for 30 minutes.

This first strand reaction product was chilled on ice before the addition of 10 □L 10×PCR buffer without Mg, 1 □L 10 mM dNTP mixture, 1.5 □L 50 mM MgCl₂, 5 □L of 10 □M primer mix, and 0.2 □L 5 U/□L Platinum Taq DNA polymerase. Autoclaved water was added to a final volume of 50 □L. The first sense primer for sialidase was 5′-CTTACAGAATCCCTGCTCTGATCTA-3′ (SEQ ID NO: 21) and the first antisense primer for sialidase was 5′-ATTTGACTCATACAGACACCCAAAT-3′ (SEQ ID NO: 22). The second sense primer for sialidase was 5′-GCCTCGGTTAAAAGTGAGAAAAG-3′ (SEQ ID NO: 23) and the second antisense primer for sialidase was 5′-AGGTAGGCTTGGGTCACCACT-3′ (SEQ ID NO: 24). Beta actin was used as a control using the sense primer 5′-AGCTGAGAGGGAAATTGTGCG-3′ (SEQ ID NO: 25) and the antisense primer 5′-GCAACGGAACCGCTCATT-3′ (SEQ ID NO: 26). The PCR reaction cocktails were transferred to a 96-well plate and the following thermal cycler program was performed. 1 cycle of (94° C.-2 min), 35 cycles of (94° C.-30 sec, 55° C.-30 sec, 72° C.-2 min). After the PCR was complete, the product was stored at 4° C. The result was verified by gel electrophoresis.

Results

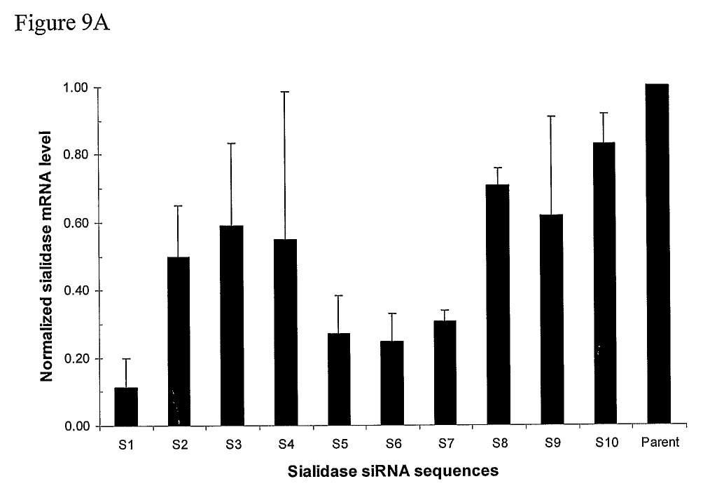

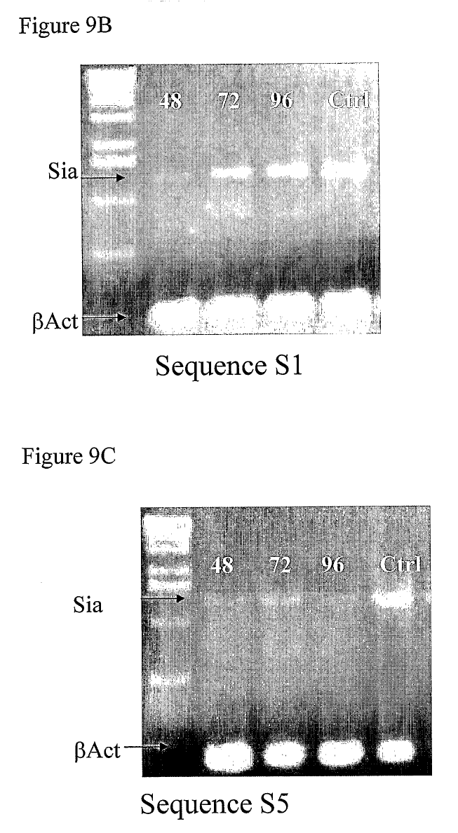

The ability of siRNAs to reduce sialidase mRNA levels was tested by transiently transfecting the siRNAs into CHO cells and performing two-step RT-PCR on total mRNA harvested 48 hours after siRNA transfection. The PCR product from the quantitative PCR assay was analyzed by agarose gel electrophoresis. FIG. 9A shows mRNA quantification from cell transfected with each of the siRNAs listed in Table 1 illustrating that mRNA levels are reduced by at least 2-fold in most cases. siRNAs S2, S3, S4, and S9 reduced sialidase mRNA levels by 2-fold (approx.). siRNAs S7 reduced sialidase mRNA levels by 3-fold (approx.). siRNAs S5 and S6 reduced sialidase mRNA by approximately 4-fold. siRNA S1 performed best, reducing sialidase mRNA levels by 9 fold.

FIGS. 9B and 9C show sialidase expression at various time points following transfection of siRNA S1 or S5, illustrating that significant silencing persists through approximately 72-96 hours but that the silencing effect diminishes over time. Similar results were obtained for siRNA S6 (data not shown).

### Example 3

### siRNAs Targeted to Sialidase Reduce Sialidase Activity

Materials and Methods

CHO cell culture and siRNA transfection were performed as described in Example 2.

Determination of Sialidase Activity

Confluent CHO cells were trypsinized using 0.05% Trypsin/EDTA solution and washed three times with PBS. 1×10⁶ cells were resuspended in cold water for osmotic lysis and were passed through 26G3/8 needles at least twenty times. 4 mM 2′-(4-methylumbelliferyl)-α-D-N-acetylneuraminic acid (4MU-NeuAc) (Sigma) diluted in potassium phosphate buffer was added to the lysate to a total volume of 100 □L. At the same time, several dilutions of sialidase (Roche) with known activities were reacted with 4MU-NeuAc as standards. The samples were incubated for 90 minutes at 37° C. after which 900 □L of 0.2M glycine buffer pH 10.4 was added to stop the enzymatic reaction. 250 □L of the final solution was transferred to 96-well black plates and fluorescence was measured using a plate reader with an excitation of 362 nm and an emission of 448 nm.

Results

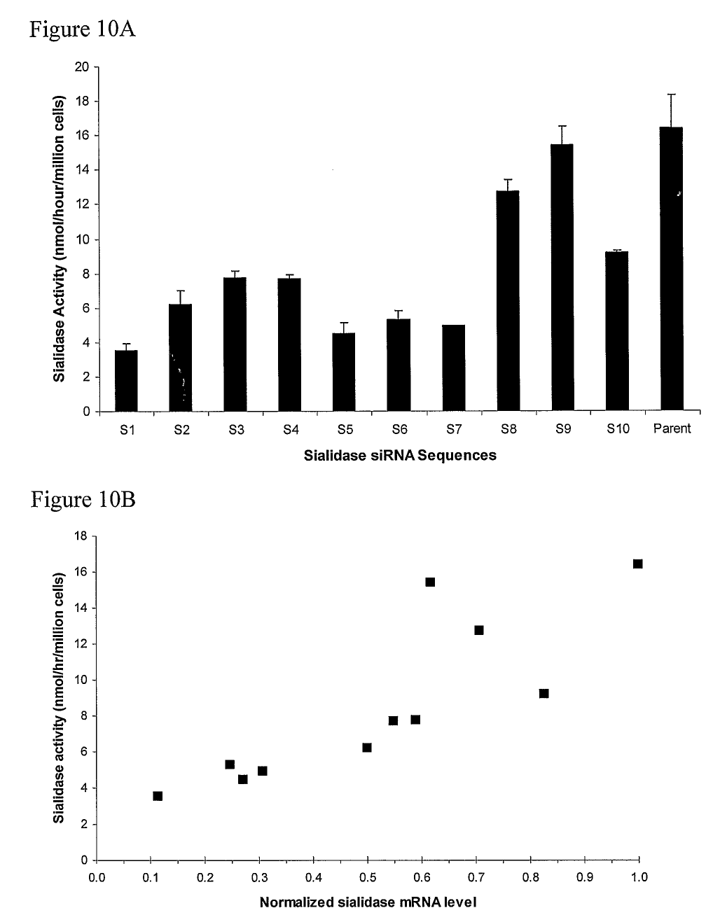

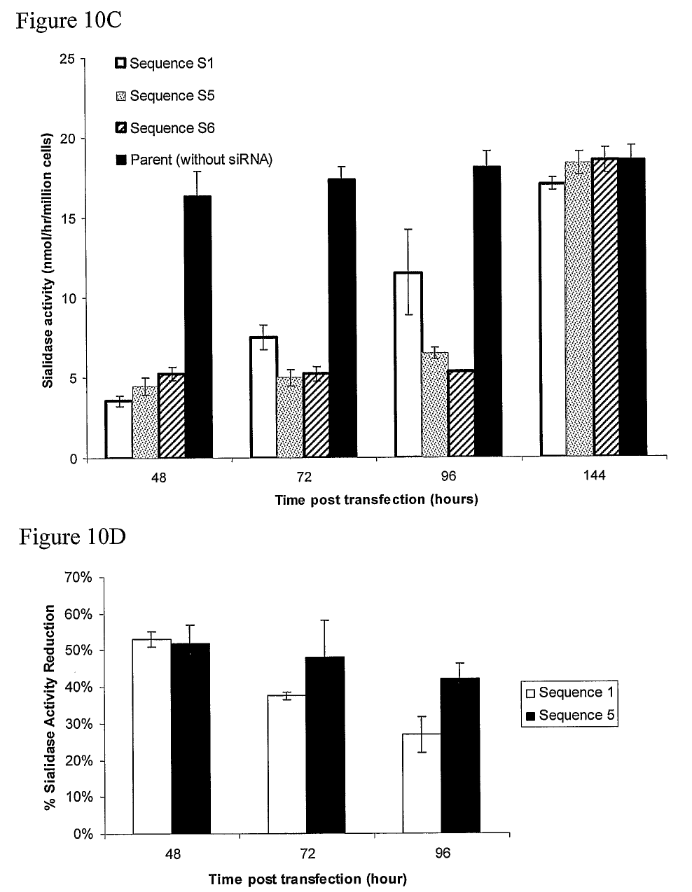

To determine whether the decrease in sialidase mRNA obtained by transfection of siRNA targeted to sialidase would be reflected in a decrease in sialidase activity, we measured sialidase activity in lysates obtained from cells harvested 48 hours after siRNA transfection. FIG. 10A shows the results of the sialidase assay, indicating that a number of the siRNAs substantially reduced sialidase activity. In almost all cases the results of the sialidase activity assay, which measures protein activity, correlated with those obtained using RT-PCR, which measures mRNA levels, i.e., greater degrees of reduction in mRNA resulted in greater reduction in sialidase activity. FIG. 10B is a plot showing that reduction in sialidase mRNA levels by RNAi corresponds to a decrease in sialidase activity.

It is worth noting the sialidase assayed for activity in this study was obtained from cell lysate. Activity assays can also be done on sialidase concentrated from supernatant. Performing activity assay on cell lysate was deemed to be a more rigorous approach as the sialidase in supernatant originated from lysate in any case. In addition, fetal bovine serum (which contains sialidase) was present in the supernatant and tended to contribute background fluorescence; hence, biasing the measurements of cell-derived sialidase activity. Even in a transformed suspension culture lacking serum, from which sialidase was isolated and concentrated using a Microsep concentrator with 10 kDA exclusion limit, the HyQPF-CHO medium contributed extremely high fluorescent background making the activity measurement unreliable. The difficulty in measuring sialidase in supernatant was also observed by others (Sung et al, 2004).

FIG. 10C shows sialidase activity at various time points following transfection of siRNAs S1, S5, or S6, illustrating that significant silencing persists through approximately 72-96 hours but that the silencing effect diminishes by 144 hours post-transfection. FIG. 10D is a plot of the same results for S1 and S5 but expresses the reduction in sialidase activity as a percent of the sialidase activity in untransfected cells.

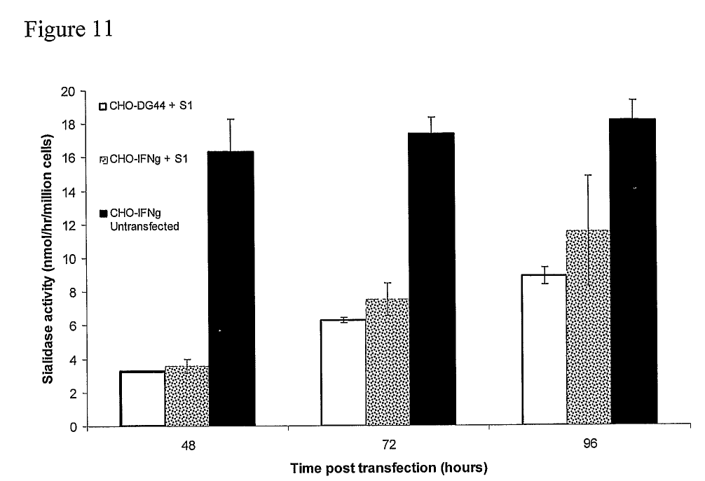

In order to show that the ability of siRNA targeted to sialidase to reduce sialidase activity was a general phenomenon, we tested siRNA S1 in a variety of other CHO cell lines, including CHO-DG44, which does not express any heterologous glycoproteins and is thus suitable as a host cell for expression of any glycoprotein of interest. S6 was transfected into the various cell lines as described above, and sialidase activity was measured in cell lysates harvested at various times after transfection. As shown in FIG. 11, siRNA S1 effectively reduced sialidase activity in all cell lines tested. The extent of inhibition was approximately the same in the different cell lines up to at least 72 hours post-transfection. In the case of all cell lines, the silencing effect diminished over time. It is likely that this transient behavior occurred because only a limited amount of siRNA was initially introduced into the culture medium, and this siRNA may have degraded over time and/or the intracellular siRNA concentration was diluted as cells divided.

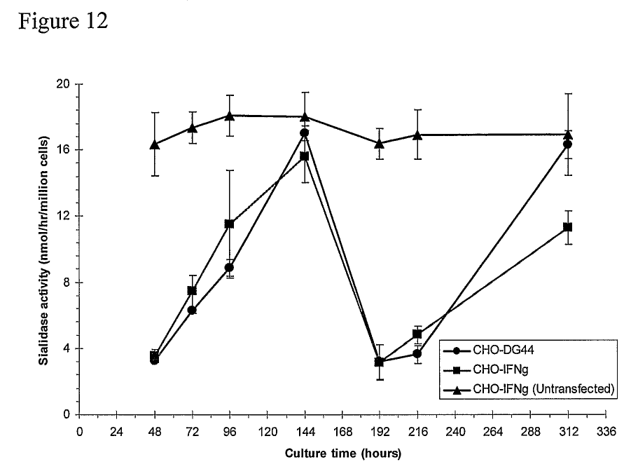

To investigate whether continuos silencing by RNAi was possible, siRNA S1 was retransfected at the time point when sialidase activity reverted to its normal level (144 hours post-transfection). The same dose of siRNA was utilized, and the same number of CHO cells (0.1 million cells) were transfected to ensure similar experimental conditions. It was observed that 48 hour post retransfection, sialidase activity was again reduced significantly with magnitudes of reduction similar to those obtained in the first transfection process (FIG. 12). However, this second transfection process also exhibited transient silencing, as it was observed that sialidase activity reverted back to its untransfected level five days post retransfection. This study demonstrated that the transient nature of siRNA transfection could be overcome by intermittently or continuously dosing the cells with an appropriate amount of siRNA.

Interestingly, the most effective sequence targets a site that was randomly selected rather than one that was selected according to the empirical rules described above. Although the empirical rules provided choices of sequences that yielded reduction in sialidase mRNA levels, a better understanding of why sequence S1 worked better despite its violation of the rules would lead to a better ability to design effective siRNAs. A number of studies have attempted to correlate siRNA sequence design with the ability of the siRNA to effectively silence its target gene (Khorova et al, 2003; Schwartz et al, 2003; Amarzguioui and Prydz, 2004; Chalk et al, 2004; Reynolds et al, 2004; Ui-Tei, 2004). For example, Khvorova and Schwartz have established a design rule based on a thermodynamic analysis derived in part from an understanding of the mechanism of RNAi.

Schwartz' asymmetrical rule and Khvorova's internal stability analysis were inspired by the understanding that one of the critical steps in RNA interference mechanism was the recruitment of anti-sense strand of siRNA duplex by RISC complex. This single-stranded antisense further guided the complex to the messenger RNA that possessed the complementary sequence, which resulted in the endonucleolytic degradation of target mRNA (Dykxhoorn et al, 2003). Thus, it was critical that the siRNA duplex unwound in a way that enabled anti-sense strand of siRNA to be recruited by RISC.

Schwartz and co-workers argued that RISC assembly favored the siRNA strand whose 5′ end was weaker in terms of binding energy. In particular, the binding energy in the antisense 5′ end must be weaker as to allow helicase to initiate unwinding process from 5′ end of antisense strand, leading to the assembly of single stranded antisense strand to RISC complex. A recruitment of sense strand into RISC complex was thought to produce no RNA interference process against the intended target.

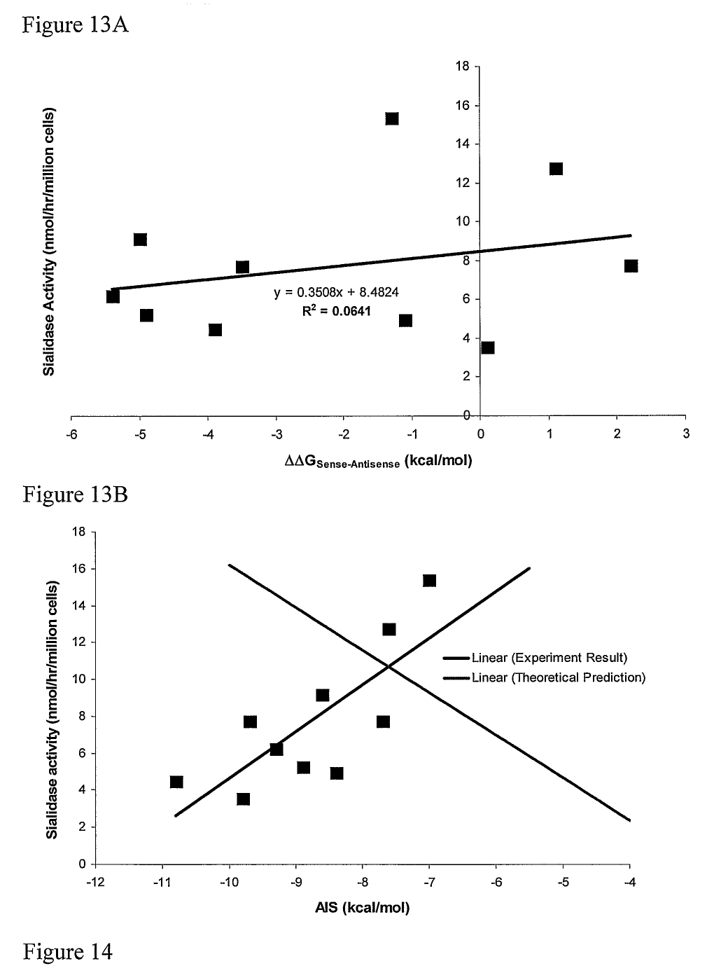

ΔGₛₑₙₛₑ and ΔGₐₙₜᵢₛₑₙₛₑ were calculated using nearest-neighbor method and the mfold algorithm. Their difference was called ΔΔG_{Sense-Antisense} whereby instability in 5′ antisense end would correspond to a ΔΔG value of less than zero. The result of the estimate is tabulated in Table 2 and the sialidase activity from the experimental results were compared to the ΔΔG estimated ala Schwartz' et al method (FIG. 13A). Although ΔΔG<0 trend was observed for some functional sialidase siRNA sequences, there was no strong correlation between the negativity of ΔΔG values and the siRNA efficacy. In fact, sequence S1, which was the best sequence found in our study, possessed positive ΔΔG values, indicating that Schwartz' asymmetry rule would not suggest the utilization of sequence S1 in knocking down sialidase activity. It would appear that the efficacy of sequence S1 efficacy was not a result of a weaker 5′ antisense strand.

Another approach to understand the reason behind the potency of S1 sequence was to employ Khvorova's average of internal stability analysis for positions 9-14 of the antisense strand (AIS) analysis. A less stable antisense strand was desired to allow initiation of helicase at 5′ antisense strand end and this would be represented by more positive AIS value. A rule of thumb established by Khvorova et al was that the AIS at positions 9-14 should be lower than −8.5 kcal/mol. This analysis was conducted on the sequences displayed in Table 1 and is shown in FIG. 13B. We found that our experimental data exhibited the opposite behavior than the ideal behavior established by Khvorova et al. In fact, sialidase siRNA sequence S1 had the most negative AIS values; an indication of stable antisense strand at position 9-14. We concluded that the best sialidase siRNA sequence potency did not originate from having an instable antisense strand as suggested by Khvorova et al. We note that sialidase siRNA sequence S1 was not in the open reading frame (ORF) region of sialidase cDNA. In fact, sequence S1 was a part of the 5′ untranslated region (UTR). It was hypothesized by Elbashir and her co-workers that UTR binding proteins might interfere with scanning process of the siRNA complex and thus, 5′ and 3′ UTR should be avoided when designing efficacious siRNA. More recent studies have indicated that siRNAs designed to target either the 5′ or 3′ UTR can effectively silence expression, as is the case with siRNA S1.

In summary, we could not explain the increased efficacy of S1 relative to the other siRNAs based on either empirical rules or thermodynamic analysis, suggesting that these rules should only be used as a guideline and emphasizing the importance of testing a number of candidate siRNAs. Table 2 summarizes the results of our thermodynamic analyses.

TABLE 2
Thermodynamic Analysis of siRNA Sequences

| | ΔG_{Antisense} | ΔG_{Sense} | ΔΔG_{Sense-Antisense} | AIS |
|---|---|---|---|---|
| Sequence | (kcal/mol) | (kcal/mol) | (kcal/mol) | (kcal/mol) |
| S1 | −8.4 | −8.3 | 0.1 | −9.8 |
| S2 | −6.1 | −11.5 | −5.4 | −9.3 |
| S3 | −9.3 | −7.1 | 2.2 | −9.7 |
| S4 | −4.9 | −8.4 | −3.5 | −7.7 |
| S5 | −7.0 | −10.9 | −3.9 | −10.8 |
| S6 | −4.9 | −9.8 | −4.9 | −8.9 |
| S7 | −7.0 | −8.1 | −1.1 | −8.4 |
| S8 | −8.3 | −7.2 | 1.1 | −7.6 |
| S9 | −4.9 | −6.2 | −1.3 | −7.0 |
| S10 | −5.9 | −10.9 | −5.0 | −8.6 |

### Example 4

### Production of Cell Lines that Express an shRNA Targeted to Sialidase

Materials and Methods

Plasmid Design and Transfection

pSilencer™ plasmids encoding a drug selection marker (Hygromycin) were obtained from Ambion. Three different plasmids, each containing a different promoter (U6, H1, or modified CMV) were utilized (Cat. Nos. 5760, 5766, and 5777, respectively). shRNA template oligonucleotides were designed based on the two sequences that most strongly silenced sialidase, as identified by RT-PCR and fluorescence assays described in Examples 2 and 3. Oligonucleotides were ordered from Integrated DNA Technologies (Coralville, Iowa). 10 ng annealed shRNA template oligonucleotides were ligated into 0.1 □g pSilencer with 5 units of T4 DNA Ligase at room temperature overnight. Ligase was inactivated by heating at 65° C. for 10 minutes. Ligation product was transformed into DH5α competent cells (Invitrogen) according to the instruction manual. A Qiagen HiSpeed Plasmid Maxi Kit was utilized to purify the plasmid. To confirm that the insert was correct and that no mutation occurred, the plasmid was sequenced with the following primer: 5′-AGGCGATTAAGTTGGGTA-3′. (SEQ ID NO: 26).

CHO-IFNγ cells were inoculated at density of 1×10⁵/well in a 6-well plate and the shRNA plasmid was linearized with XmnI (New England Biolabs) one day before the transfection. 3 □L Fugene 6 (Roche) was added to 97 □L serum-free DMEM. 1 □g of linearized plasmid was gently mixed with the diluted Fugene and the final mixture was incubated for 30 minutes at room temperature before being added to CHO-IFNγ. 16 hours later the medium was replaced with DMEM supplemented with 600 □g/mL Hygromycin. The medium was changed every 3 days and when the confluence reached >80%, the cells were split. Cloning rings were used to isolate numerous cell colonies which were then transferred to a 24 well plate. When the confluency reached >80%, the colonies were moved to 6 well plates.

Screening for Low Sialidase Activity

1×10⁶ cells from each clone were lysed using the procedures outlined above and were tested for sialidase activity as described in Example 3. Clones with sialidase activity reduced by 50% or greater were passaged a second time and re-assayed. Some clones did not exhibit consistent reduction and were discarded. The remaining clones were passaged and re-assayed. The best three clones were further characterized.

Results

We next desired to establish cell lines in which sialidase expression would be silenced in a more long-lasting manner. To this end, we designed shRNAs based on the sequences of the most effective siRNAs (S1 and S5) and engineered cells that express them. Such cells continuously produce shRNA, which is further processed intracellularly into siRNAs targeted to sialidase. The shRNAs were designed to include both antisense and sense sequences as present in S1 or S5, separated by a 9 nt spacer region predicted to form a loop. The complete sequences of the oligonucleotides that encode the two shRNAs are shown in Table 3, in which “sense” indicates the sequence of an oligonucleotide that incorporates sense and antisense portions that would, in RNA form, self-hybridize to produce an shRNA targeted to sialidase and “antisense” indicates an oligonucleotide with portions complementary to the sense, antisense, and loop regions of such an shRNA. The sequences in italics represent recognition sites for BamHI and HindIII.

TABLE 3
shRNA Sequences

| | |
|---|---|
| Description | Sequence |

| | |
|---|---|
| Sequence S1 | 5′-GATCC ATCATCTGCAGGGCCTGGG TTCAA |
| shRNA Sense | GAGACCGAGGCCCTGCAGATGATCC A-3′ |
| | (SEQ ID NO: 27) |
| Sequence S1 | 5′-AGCTT GGATCATCTGCAGGGCCTCGG TCT |
| shRNA Antisense | CTTGAACCGAGGCCCTGCAGATGAT G-3′ |
| | (SEQ ID NO: 28) |
| Sequence S5 | 5′-GATCC GCCGGTCCTCCCTTCTCCA TTCAA |
| shRNA Sense | GAGATGGAGAAGGGAGGACCGGCTT A-3′ |
| | (SEQ ID NO: 29) |
| Sequence S5 | 5′-AGCTT AAGCCGGTCCTCCCTTCTCCA TCT |
| shRNA Antisense | CTTGAATGGAGAAGGGAGGACCGGC G-3′ |
| | (SEQ ID NO: 30) |

We integrated the two sequences into plasmids that once transfected into the cell would result in continuous production of shRNA. The shRNAs are expected to have the following sequence for S1: 5′-ATCATCTGCAGGGCCTCGG TTCAAGAGA CCGAGGCCCTGCAGATGATCC-3′ (SEQ ID NO: 31), and the following sequence for S5: 5′-GCCGGTCCTCCCTTCTCCA TTCAAGAGA TGGAGAAGGGAGGACCGGCTT-3′ (SEQ ID NO: 32). The pSilencer plasmids utilizing the pol III promoters, U6 and H1 (Ambion), were initially used to drive the expression of sialidase shRNA. Screening was a significant obstacle because drug resistance (resulting from the hygromycin resistance gene in the plasmid) did not guarantee that a high number of copies of the plasmid were integrated into the genome in each CHO cell. Nearly 1000 clones were tested, and we were unable to obtain cell lines with over 30% reduction in sialidase activity reduction using these U6 and H1 promoter-incorporating plasmids. Four clones were found to have about 15-20% sialidase activity reduction (data not shown). Unfortunately, the sialidase knockdown was not maintained after the cells were passaged numerous times.

We therefore turned to a Pol II promoter in an effort to obtain greater levels of stable reduction in sialidase activity. Specifically, a modified CMV promoter was utilized to drive the long-term expression of sialidase siRNA (Foecking and Hofstetter, 1986; Xia et al, 2002). Initially, out of 300 clones generated by transfection of the modified CMV plasmid, three clones were found to reduce sialidase activity by three fold. These clones were further analyzed by growing them for longer periods of time in batch mode. Clone S1E (which has plasmid encoding sequence S1) and clone S5F (which contains the plasmid encoding sequence S5) were found to maintain low sialidase activity as compared to the parent cell throughout the cell culture even when the cells began to die (FIG. 14). The low sialidase activity was observed consistently during the lag phase, growth phase, stationary phase, and death phase of the cell culture. However, clone S5B (which contains the plasmid encoding sequence S5) did not display a consistent reduction in sialidase activity level. After 24 hours (during growth phase), clone S5B slowly exhibited sialidase activity close to the parent cell line and after 72 hours (during stationary phase), it exhibited sialidase activity close to the clone S1E. During the death phase of cell culture, clone S5B's sialidase activity increased to the level close to the parent cell line. Due to its inconsistency of sialidase activity, we did not further analyze clone S5B.

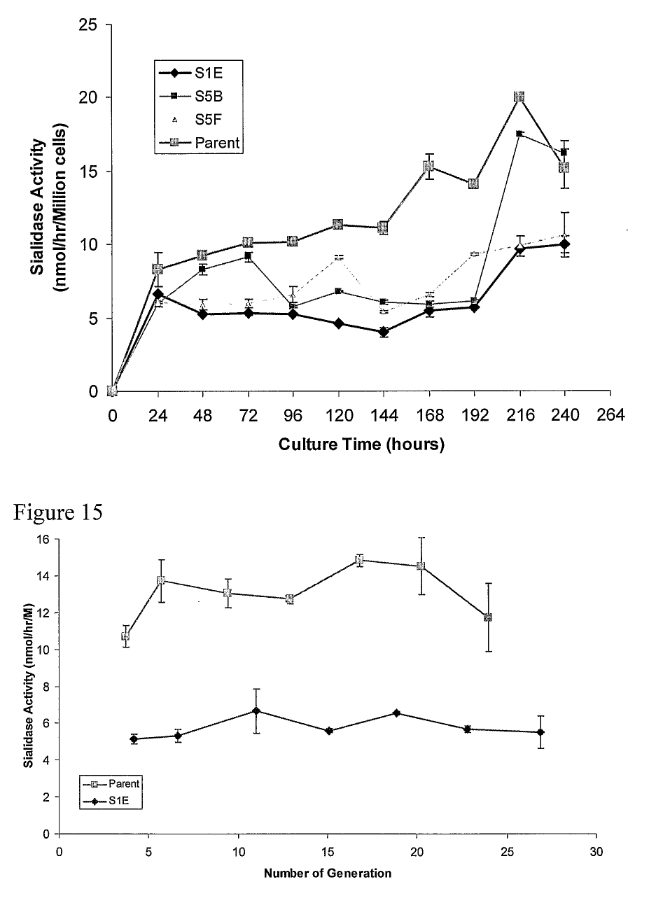

We wanted to confirm that sialidase activity would remain reduced over multiple generations as would be highly desirable for use of this approach for production of glycoproteins. Cell lines in which sialidase activity reverted to the activity of untransfected CHO cells would be less useful because increased sialidase activity would correspond to lower sialic acid content of the glycoprotein product. We therefore analyzed S1E cell lines and parental cell lines during their log growth phase from generation to generation. It was found that the reduction in sialidase activity possessed by S1E cell lines continued to exist after 25 generations (FIG. 15).

### Example 5

### Stable Expression of shRNA Targeted to Sialidase Allows High Cell Viability and Glycoprotein Production

In addition to confirming that sialidase activity could be stably silenced over multiple generations, we explored the effect of the silencing on cell viability. siRNAs were used to decrease sialidase activity in part because different levels of silencing can be obtained by selecting different siRNA sequences, so that a potentially lethal effect of complete knockout could be avoided. One study performed on rat myoblast L6 cells showed that sialidase played a role in differentiation (Sato and Miyagi, 1996), suggesting that the protein may play a role in maintaining cell viability. Reduced cell viability may have undesirable effects on glycoprotein production. We therefore performed cell viability analysis to determine whether the cells with stably reduced sialidase activity would display normal cell growth.

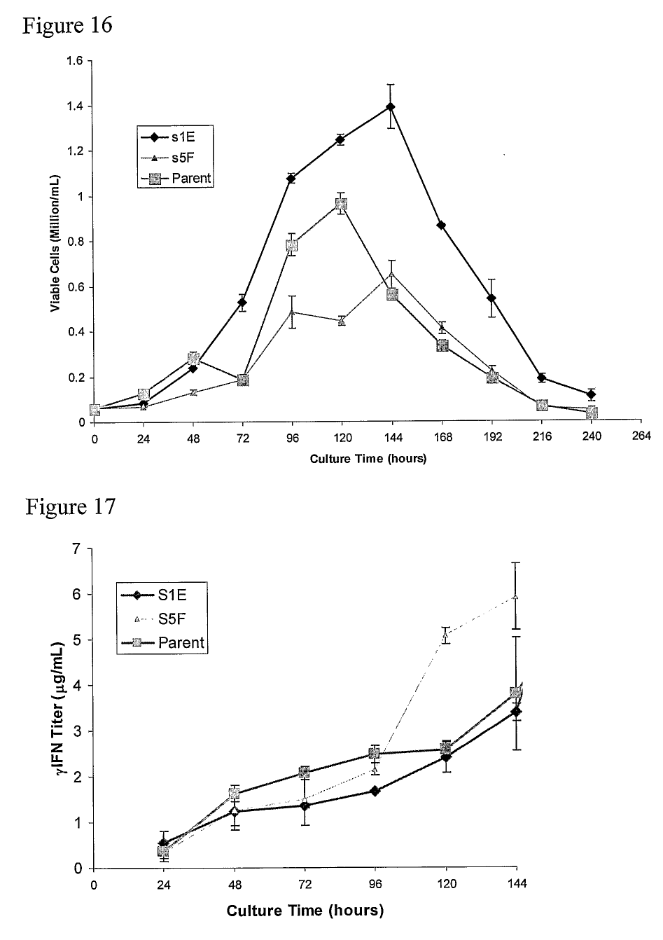

Clones S1E and S5B were compared with parent cell lines during 2 weeks of batch culture. Both clones exhibited similar growth profiles as shown by similar lengths of lag phases, exponential growth phases, and death phases (FIG. 16). It was observed that clone S1E exhibited a maximal cell density 40% higher than parent cell lines, while clone S1B exhibited a maximal cell density 40% lower than parent cell lines. Based on this data, it appears that reducing sialidase activity is consistent with maintaining high cell viability, possibly even exceeding that of cells with normal sialidase expression.

Another important issue to consider is the titer of the glycoprotein (IFNγ) produced from the CHO cells. Plasmid integration occurs randomly within the genome and although the probability of integration in the IFNγ site was very low, it is important to ensure that the cell continues to produce glycoprotein at acceptable levels. ELISA (Biosource International Inc) was used to quantify IFNγ titer during exponential growth phase indicated that the titer in each clone was comparable to parent cell lines (FIG. 17).

### Example 6

### Interferon Produced by Cells that Express an shRNA Targeted to Sialidase Exhibits Increased Sialic Acid Content

Materials and Methods

Determination of IFN-γ Concentration

IFN-γ concentrations were measured using a commercially available ELISA kit (Biosource International Inc.).

Purification and Quantification of IFN-γ

Supernatant from stable clones and untransfected parent CHO-IFNγ was collected at various time points during cell culture. The supernatant was centrifuged at 1000 rpm for 10 minutes and filtered (0.22 □m). 50 mL of prepared supernatant was loaded at 0.2 mL/min onto an anti-human IFNγ immunoaffinity column that had been equilibrated with loading buffer (20 mM sodium phosphate buffer and 150 mM sodium chloride adjusted to pH 7.2). This purification process was performed on an AKTA Explorer™ 100 chromatographic system (Amersham Biosciences, Uppsala, Sweden). The column was then washed with loading buffer and the sample was eluted using elution buffer at 0.02 mL/min. The elution buffer consisted of 150 mM sodium chloride adjusted with HCl to pH 2.5. After eluting the samples, the column was regenerated for subsequent runs using loading buffer. Reverse phase HPLC was performed to quantify purified IFN.\tilde{γ}25-50 □L purified IFNγ was injected into a Shimadzu LC-10ADvp HPLC (Shimadzu Analytical instruments, Kyoto, Japan) and separated on a Vydac C18 1 mm×250 mm column (Grace Vydac, Hesperia, Calif.). The sample was eluted over a 30 min linear gradient from 35% (v/v) to 65% (v/v) buffer B (buffer A: HPLC grade water+0.1% trifluoroacetic acid (TFA); buffer B: HPLC grade acetonitrile+0.1% TFA) at a flow rate of 0.05 ml/min. Eluted IFNγ was monitored at 220 nm and quantified by comparing the samples with standards of known IFN-γ concentration.

Sialic Acid Content Analysis of IFN-γ

A modified version of the thiobarbituric acid assay (TAA) was used to measure the amount of total sialic acid per protein (Hammond and Papermaster, 1976). Sialic acid from 3-6 □g of purified IFN-γ was cleaved by incubating IFN-γ with 0.0025 U sialidase (Roche) for 24 hours at 37 C. After digestion, water was added to yield a final volume of 500 □L. 250 □L of periodic acid reagent (25 mM periodic acid in 0.125NH₂SO₄) was added to this mixture which was then incubated at 37 C for 30 minutes. Excess periodate was destroyed by adding arsenite solution (200 □L of 2% sodium arsenite in 0.5N HCl) before 2 mL of thiobarbituric acid reagent (2 mL of 0.1M 2-thiobarbituric acid, adjusted to pH 9 with NaOH) was added. The solution was heated at 98 C for 7.5 min then incubated on ice for 10 minutes. 1.5 mL of acid/butanol solution (n-butanol with 5% (v/v) 12N HCl) was added to the cooled sample and the mixture was shaken vigorously before it was centrifuged at 3,000 rpm for 3 minutes. The clear organic phase was transferred to a 10 mm cuvette and the fluorescence intensity (□ₑₓ=550 nm, □ₑₘ=570 nm) was measured with a Cary Eclipse Fluorescence Spectrophotometer (Varian Inc, Palo Alto, Calif.). Standard curve generated from pure sialic acid samples was used to quantify the sialic acid content from each sample. The assay was repeated three times.

Results

We next sought to examine the important question of whether lower levels of sialidase activity result in improved in sialic acid content of the glycoprotein throughout cell culture. Of particular interest is the sialic acid content during the death period when cytosolic sialidase is released into the culture medium (Gramer et al, 1995; Munzert et al, 1996; Gu et al, 1997; Gramer, 2000).

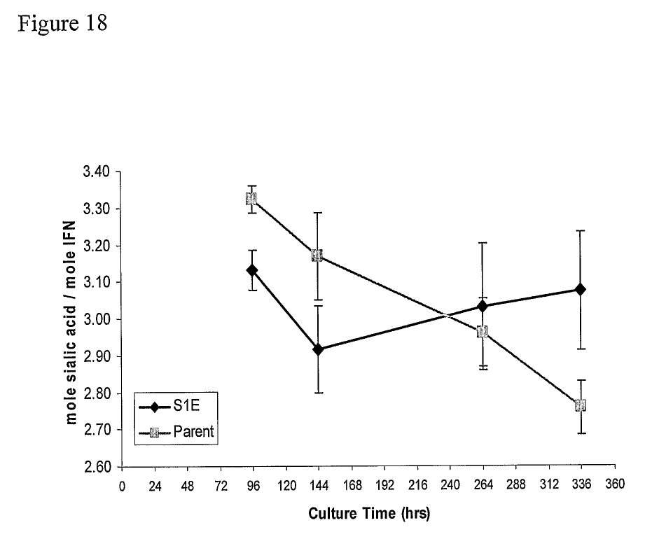

To address this issue, IFNγ was collected and purified from two distinct phases: growth phase, when the cells were still actively growing, and death phase, when nutrient depletion occurred and the cells began to die. As shown in FIG. 18, after 96 hours of culture (which is still within the growth phase of the cells), parental cells maintained high sialic acid content per IFNγ molecule, 3.13 mole sialic acid/mole IFNγ. However, as the cells began to die, sialic acid content decreased significantly at a rate of 0.05 moles of sialic acid/moles of IFN.γ loss/day as previously reported. This decrease is believed to result from the release of cytosolic sialidase into the culture supernatant which in turn cleaves the sialic acid from IFNγ.

In contrast, this phenomenon did not occur with clone S1E. In fact, sialic acid remained at roughly 3-3.1 mole sialic acid/mole IFNγ during both growth phase and death phase, thereby demonstrating the effectiveness of siRNA in knocking down sialidase activity and maintaining sialic acid content throughout the cell culture period. It is worth noting that at 144 hours (which was the transition period from growth phase to death phase), the sialic acid content of IFNγ from S1E clones went down to about 2.9 mole sialic acid/mole IFNγ before returning to the average value of 3.1 mole sialic acid/mole IFNγ. This result can be explained by recognizing that at the onset of cell death, residual cytosolic sialidase was released into the supernatant. Although sialidase siRNA reduced sialidase activity by interfering with sialidase expression, it did not completely eliminate sialidase. With a cytosolic sialidase half life of about 57 hours at 37° C., residual sialidase released in the supernatant would still be active in cleaving sialic acid off of IFNγ glycans (Gramer and Goochee, 1993). As culture time increased, residual sialidase was no longer active and a steady state value of 3.1 mole sialic acid/mole IFN.γ was observed. This result is comparable with the relatively constant sialic acid profile obtained when the sialidase inhibitor (2,3-dehydro-2-deoxy-N-acetylneuraminic acid) was added to the cell supernatant (Gu et al, 1997).

### Example 7

### Sialidase Inhibition by RNAi does not Alter the Glycan Site Distribution of IFNγ

Materials and Methods

Glycan Site Occupancy Analysis of IFN-γ

Site occupancy analysis of three IFN-γ glycoforms was performed using Micellar Electrokinetic Capilary Chromatography (MEKC) on a Beckman Coulter P/ACE™ MDQ capillary electrophoresis system (Beckman Coulter, Fullteron, Calif.). A 50 □m diameter×52 cm (40 cm length to detector) unfused silica capillary (Beckman Coulter) was used for the separation. The capillary was initially cleaned with 0.1M NaOH for 15 minutes, flushed with HPLC grade water for 10 minutes, and subsequently equilibrated with running buffer for 15 minutes. The running buffer consisted of 30 mM sodium borate, 30 mM boric acid and 100 mM SDS at pH 9. Samples were pressure injected at 3 psi over 10 second and then 12-15 kV voltage was applied to the capillary over 60-80 minutes. The chromatograms were integrated to quantify the percentage of 2-site, 1-site, and nonglycosylated peaks.

Results

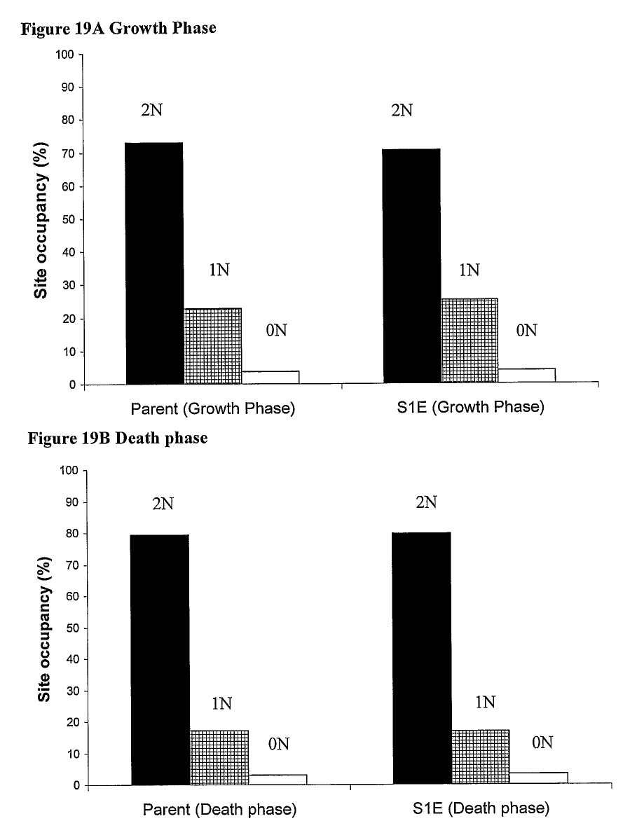

Another set of studies explored the distribution of glycoforms throughout the cell culture. As shown in FIG. 19, glycan site occupancy of IFNγ remained relatively unchanged when sialidase activity was reduced. This phenomenon was also observed throughout the various phases of cell culture (i.e. growth phase and death phase). This study indicated that sialidase inhibition did not significantly alter the glycan site distribution of IFNγ, at least not within the limits of detection of this assay and over the time period studied.

### REFERENCES

Borys, M. C., Linzer, D. I. H., and Papoutsakis, E. T. 1993. Culture pH Affects Expression Rates and Glycosylation of Recombinant Mouse Placental Lactogen Proteins by CHO Cells. Biotechnology, 11:720-724.
Chotigeat, W., Watanapokasin, Y., Mahler, S., Gray, P. P., 1994. Role of environmental conditions on the expression levels, glycoform pattern and levels of sialyltransferase for hFSH produced by recombinant CHO cells. Cytotechnology 15, 217-221.
Chung, B. S., Jeong, Y. T., Chang, K. H., Kim, J. S., Kim, J. H., 2001. Effect of sodium butyrate on glycosylation of recombinant erythropoietin. J. Microbiol. Biotechnol. 11, 1087-1092.
Dykxhoorn, D. M., Novina, C. D., Sharp, P. A. 2003. Killing the messenger: short RNAs that silence gene expression. Nature Reviews Molecular Cell Biology. 4: 457-467.
Elbashir, S. M., Harborth, J., Lendeckel, W., Yalcin, A., Weber, K. and Tuschl, T. 2001a. Duplexes of 21-nucleotide RNAs Mediate RNA Interference in Cultured Mammalian Cells. Nature. 411:494-498.
Elbashir, S. M., Lendeckel, W. and Tuschl, T. 2001b. RNA Interference is Mediated by 21- and 22-nucleotide RNAs. Genes and Development, 15: 188-200.
Elbashir, S. M., Martinez J., Patkaniowska, A., Lendeckel, W. and Tuschl, T. (2001c). Functional anatomy of siRNAs for mediating efficient RNAi in Drosophila melanogaster embryo lysate. European Molecular Biology Organization Journal, 20, 6877-6888.
Ferrari, J., et al., (1994). Glycobiology, 4: 367-373.
Foecking M K, Hofstetter H (1986) Powerful and versatile enhancer-promoter unit for mammalian expression vectors. Gene 145 (1): 101-5.
Goldwasser, E., Kung, C. K. H., Eliason, J. 1974. On the Mechanism of Erythropoietin-Induced Differentiation.XIII The Role of Sialic Acid in Erythropoietin Action. J. Biol. Chem. 249: 4202-4206.
Gramer, M. J. and Goochee, C. F. 1993. Glycosidase Activities in Chinese Hamster Ovary Cell Lysate and Cell Culture Supernatant. Biotechnol. Bioeng., 9: 366-373.
Gramer, M. J., Goochee, C. F., Chock, V. Y., Brousseau, D. T., and Slikowski, M. B. 1995. Removal of Sialic Acid from a Glycoprotein in CHO Cell Culture Supernatant by Action of an Extracellular CHO Cell Sialidase. Biotechnology, 13: 692-698.
Gramer, M. J. 2000. Detecting and Minimizing Glycosidase Activities that Can Hydrolize Sugars from Cell Culture-Produced Glycoproteins. Molecular Biotechnology, 15:69-75.
Gu, X., Harmon, B. J., and Wang, D. I. C. 1997. Site- and Branch-Specific Sialylation of Recombinant Human Interferon-g in Chinese Hamster Ovary Cell Culture. Biotechnol. Bioeng., 55: 390-398.
Hayter, P. M., Curling, E. M., Baines, A. J., Jenkins, N., Salmon, I., Strange, P. G., Tong, J. M. and Bull, A. T. 1992. Glucose-Limited Chemostat Culture of CHO Cells Producing Recombinant Human Interferon-γ. Biotechnol. Bioeng., 39:327-335.
Kimura, R. and Miller, W. M. 1997. Glycosylation of CHO-Derived tPA Produced under Elevated pCO₂. Biotechnol. Prog. 13:311-317.
Kwon, Ki-Sun; Yu, Myeong-Hee. 1997. Effect of glycosylation on the stability of α1-antitrypsin toward urea denaturation and thermal deactivation. Biochimica et Biophysica Acta. 1335(3): 265-272
Lawson, E. Q., Hedlund, B. E., Ericson, M. E., Mood, D. A., Litman, G. W., Middaugh, R. 1983. Effect of carbohydrate on protein solubility. Arch. Biochem. Biophys. 220: 572-575.
Miyagi, T., et al., 1993. Molecular cloning and expression of cDNA encoding rat skeletal muscle cytosolic sialidase. J. Biol. Chem. 268: 26435-26440.
Munzert, E., Muthing, J., Buntemeyer, H., Lehmann, J. 1996. Sialidase activity in culture fluid of chinese hamster ovary cells during batch culture and its effect on recombinant human antithrombin III integrity. Biotechnol. Prog. 12: 559-563.
Novina, C. D., Sharp, P. A. 2004. The RNAi revolution. Nature. 430: 161-164.
Oh-Eda, M., Hasegawa, M., Hattori, K., Kuboniwa, H., Kojima, T., Orita, T., Tomonou, K., Yamazaki, T. and Ochi, N. 1990. O-linked Sugar Chain of Human G-CSF Protects it Against Polymerization and Denaturation Allowing it to Retain its Biological Activity. J. Biol. Chem., 265:11432-11435.
Reynolds A, Leake D, Boese Q, Scaringe S, Marshall W S, Khvorova A. 2004. Rational siRNA design for RNA interference. Nat. Biotechnol. 22(3):326-30.
Sato, K., Miyagi, T. 1996. Involvement of an endogeneous sialidase in skeletal muscle cell differentiation. Biochem. Biophys. Res. Commun. 221: 826-830.
Schwarz D S, Hutvagner G, Du T, Xu Z, Aronin N, Zamore P D. 2003. Asymmetry in the assembly of the RNAi enzyme complex. Cell. 115(2):199-208, 2003.
Smith, P. L., Bousfield, G. R., Kumar, S., Fiete, D. and Baenziger, J. U. 1993. Equine Lutropin and Chorionic Gonadotropin Bear Oligosaccharides Terminating with SO₄-4-GalNAc and Sia-α 2,3 Gal, Respectively. J. Biol. Chem., 268:795-802.
Sung, Y. H., Song, Y. J., Lim, S. W., Chung, J. Y., and Lee, G. M. 2004. Effect of sodium butyrate on the production, heterogeneity, and biological activity of human thrombopoietin by recombinant Chinese hamster ovary cells. Journal of Biotechnology. 112: 323-335
Wittwer, A. J., and Howard, S. C. 1990. Glycosylation at Asn-184 Inhibits the Conversion of Single-Chain to Two-Chain Tissue-Type Plasminogen Activator by Plasmin. Biochemistry, 29:4175-4180.
Weiss, P., Ashwell, G. 1989. The asialyglycoprotein receptor: properties and modulation by ligand. Prog. Clin. Biol. Res. 300: 169-184.
Wright, T. H. 1991. Non enzymatic deamidation of aspraginyl and glutaminyl residues in proteins. Critical Rev. Biochem. Mol. Biol. 26: 1-52.
Xia H, Mao Q, Paulson H, Davidson B (2002) siRNA-mediated gene silencing in vitro and in vivo. Nature Biotechnology 20: 1006-10
Zanghi, J. A., Schmelzer, A. E., Mendoza, T. P., Knop, R. H., Miller, W. M. 1999. Bicarbonate Concentrations and Osmolality are Key Determinants in the Inhibition of CHO Cell Polysialylation under Elevated pCO₂ or pH. Biotechnol. Bioeng., 65:182-191.

1. An RNAi agent targeted to a gene that encodes sialidase.

2-12. (canceled)

13. The RNAi agent of claim 1, wherein the RNAi agent reduces expression of sialidase mRNA by between 10-fold and 100-fold when present in a cell.

14. The RNAi agent of claim 1, wherein the RNAi agent reduces expression of sialidase mRNA by between 100 and 1000-fold when present in the cell.

15. The RNAi agent of claim 1, wherein the RNAi agent reduces expression of sialidase mRNA by between 1000 and 10000-fold when present in the cell.

16. The RNAi agent of claim 1, wherein the RNAi agent reduces sialidase activity by between approximately 20% and 60% when present in a cell.

17. (canceled)

18. The RNAi agent of claim 1, wherein the RNAi agent is targeted to a portion of the sialidase gene whose sequence is selected from the group consisting of N1-N19 of any of SEQ ID NOs: 1, 3, 5, 7, 9, 11, 13, 15, 17, and 19.

19. The RNAi agent of claim 1, wherein the RNAi agent comprises or encodes an antisense strand whose sequence is selected from the group consisting of N1-N19 of any of SEQ ID NOs: 2, 4, 6, 8, 10, 12, 14, 16, 18, and 20.

20. The RNAi agent of claim 1, wherein the RNAi agent comprises or encodes a sequence selected from the group consisting SEQ ID NOs: 31 and 32.

21. A cell comprising the RNAi agent of claim 1.

22-43. (canceled)

44. A method of reducing sialidase activity in a cell comprising contacting the cell with an RNAi agent targeted to a gene that encodes sialidase.

45-67. (canceled)

68. A method of generating a cell line comprising steps of:
(a) contacting a cell with an RNAi expression vector under conditions suitable for uptake of the vector;
(b) isolating single cells that have taken up the vector; and
(c) screening populations of cells derived from the single cells of step (b) to identify cells that display reduced sialidase activity relative to parental cells.

69. A method of producing a glycoprotein comprising steps of:
(a) providing a cell line that expresses a glycoprotein of interest and expresses an RNAi agent targeted to a gene that encodes sialidase;
(b) maintaining the cell line for a period of time under conditions suitable for cell growth; and
(c) harvesting the glycoprotein of interest.

70. The method of claim 69, wherein the RNAi agent is an siRNA.

71. The method of claim 69, wherein the RNAi agent is an shRNA.

72. The method of claim 69, wherein the cell line stably expresses the RNAi agent.

73. The method of claim 69, wherein the cell line stably expresses the RNAi agent.

74. (canceled)

75. The method of claim 69, wherein the cell line has reduced sialidase activity relative to parental cells and wherein the reduced sialidase activity is maintained over 25 generations.

76. The method of claim 69, wherein the cell line is a mammalian cell line.

77. The method of claim 69, wherein the cell line is a mouse, rat, human, or non-human primate cell line.

78. The method of claim 69, wherein the cell line is a CHO cell line.

79. The method of claim 69, wherein step (c) comprises harvesting cells.

80. The method of claim 69, wherein step (c) comprises harvesting tissue culture medium.

81. The method of claim 69, wherein the glycoprotein is a therapeutic glycoprotein.

82. The method of claim 69, wherein the glycoprotein is selected from the group consisting of: immunoglobulins, antibodies, hormones, cytokines, blood clotting factors, and growth factors.

83. The method of claim 69, further comprising the step of purifying the glycoprotein of interest.

84-85. (canceled)
